(19) United States
(12) Patent Application Publication
(10) Pub. No.: US 2026/0350742 A1
(43) Pub. Date: Nov. 5, 2026

(54) METHOD FOR STITCHING OVERLAPPING TILES AND SWATHS OF GENOMIC SEQUENCE DATA

(71) Applicant: IILLUMINA, INC, San Diego, CA (US)

(72) Inventors: Milana Gataric, Cambridge (GB); Sujai Chari, Burlingame, CA (US); Jeffrey Fisher, San Diego, CA (US); Christopher Ediund, Carlsbad, CA (US); Sven Bilke, Lemon Grove, CA (US)

(21) Appl. No.: 19/485,570

(22) PCT Filed: Feb. 21, 2025
(86) PCT No.: PCT/US2025/016774

§ 371 Date: Nov. 18, 2025

Related U.S. Application Data
(60) Provisional application No. 63/558,549, filed on Feb. 27, 2024.
(60) Provisional application No. 63/558,918, filed on Feb. 28, 2024.

(51) Int. Cl. G06T 3/4038; G06T 3/02; G06T 3/14; G16B 20/20; G16B 30/20

(52) U.S. Cl. CPC G06T 3/4038; G06T 3/02; G06T 3/14; G16B 20/20; G16B 30/20

(57) ABSTRACT

Systems and methods are described herein for stitching genomic sequence data. For respective pairs of overlapping swaths of genomic sequence data, a computing device obtains tile data for respective tiles in the pair of overlapping swaths. For respective pairs of adjacent tiles in the pair of overlapping swaths, the computing device identifies one or more matching reads at respective locations within the pair of adjacent tiles, determines a pairwise offset for the pair of adjacent tiles based on the respective locations of the matching read(s), and determines a swath offset for the pair of overlapping swaths based on the pairwise offsets for the respective pairs of adjacent tiles in the pair of overlapping swaths. The computing device stitches respective pairs of adjacent tiles together by aligning coordinate systems of respective tiles using the swath offset to generate a continuous coordinate system across the pair of overlapping swaths.

### CROSS-REFERENCE TO RELATED APPLICATIONS

This application claims priority to and the benefit of the (1) U.S. Provisional Patent Application No. 63/558,549 entitled “Quality Control of Spatial Analysis products for Spatial Genomics Sequencing” filed on Feb. 27, 2024, and (2) U.S. Provisional Patent Application No. 63/558,918 entitled “Method for Stitching Overlapping Tiles and Swaths of Genomic Sequence Data,” filed on Feb. 28, 2024, the entire disclosures of each of which are hereby expressly incorporated by reference.

### FIELD OF THE INVENTION

The present disclosure generally relates to the field of genomic sequencing and quality control systems, and in particular, to stitching overlapping tiles and/or swaths of genomic sequence data, and computer-implemented methods and systems for improving quality control of substrate analysis products.

### BACKGROUND

In conventional flow cell sequencing and processing, a flow cell is sequenced and processed in strips, referred to as swaths, with a horizontal gap (e.g., 100 μm gaps) between neighboring swaths. Thus, portions of the flow cell are not detected which reduces yield and negatively impacts the base-calling quality. These swaths may be combined for example, using visual characteristics of images of the flow cell. Furthermore, the swaths are typically divided into tiles, and the tiles are processed using real-time analysis (RTA). However, in addition to horizontal gaps, the RTA typically creates vertical gaps (e.g., 10 μm gaps) between adjacent tiles, further reducing the yield and negatively impacting the base-calling quality.

In spatial genomics sequencing, the generation of spatial analytics products (e.g., spatial molecular maps) in downstream processes relies on a uniform distribution of spatial information in upstream spatial analysis products/processes. For example, the quality of spatial molecular maps produced in a downstream sequencing process (second seq) relies on a uniform distribution of spatial barcodes across flow cells in a first sequencing process (first seq). For example, regions of variable cluster density and quality in upstream processes can lead to inferior spatial analytics products (e.g., nonuniform spatial sensitivity can lead to damaged spatial molecular maps with various local artifacts such as scratches, bubbles, etc., undermining ultimate product quality). At the same time, sequencing instruments produce large numbers of raw reads (millions, billions or more per sequencing run) with a significant proportion of these being invalid for further analysis.

Accordingly, there is a need for platforms and techniques for performing improved quality control of spatial analysis products/processes, to facilitate usable and reliable downstream spatial analysis products/processes.

### SUMMARY

To address the limitations in conventional flow cell sequencing and processing, a tile and swath stitching system is described herein. The tile and swath stitching system may process the genomic sequence data using overlapping tiles and/or swaths. For example, the coordinates of the swaths may have a 3% overlap along the horizontal axis. The tile and swath stitching system may then stitch the swaths together, for example after performing the RTA of the flow cell. More specifically, the tile and swath stitching system may divide the swaths into tiles and identify reads within overlapping tiles. The tile and swath stitching system may then identify clusters of reads with matching sequences in adjacent, overlapping tiles along the horizontal direction. The tile and swath stitching system may use the locations of the matching sequences within the respective tiles to align the overlapping tiles and, for example, generate a stitched area which may include the adjacent tiles. Accordingly, the tiles may be stitched together in an image-less manner. Instead of using visual characteristics of images to determine where and how to stitch the tiles together, the tiles are stitched after identifying genomic sequence data within the respective tiles based on the coordinate locations of matching sequences identified within the respective tiles.

Then the tile and swath stitching system may filter out the duplicate reads from the stitched area. For example, the tile and swath stitching system may determine the midpoint of the stitched area and may filter out reads on the opposite side of the midpoint from the tile in which they were originally identified. The tile and swath stitching system may repeat this process for each pair of adjacent, overlapping tiles along the horizontal direction, thereby removing the horizontal gaps between the tiles with minimal duplicate data.

In addition to removing horizontal gaps, the tile and swath stitching system may remove vertical gaps between tiles. To remove vertical gaps between the tiles, the swaths may be divided into a set of tiles that overlap vertically. Then the tile and swath stitching system may stitch tiles in the vertical direction by identifying adjacent, overlapping tiles along the vertical direction, and using the locations of clusters with matching sequences, align the tiles and stitch them together.

Accordingly, the tile and swath stitching system may generate a spatial molecular map of genomic data without any horizontal or vertical gaps, thereby improving base-calling quality and generating a continuous coordinate system for each of the clusters in a flow cell. In fact, stitching tiles and/or swaths together may be one of the few, if not the only way, to produce a gap-less continuous coordinate system for a spatial molecular map when a flow cell is used as a substrate for spatial sequencing. Furthermore, it should be appreciated that the tile and swath stitching system described herein increases the yield of the genomic sequence data, thereby recovering genomic reads that would have otherwise been lost under the conventional systems described above.

In some implementations, the tile and swath stitching system may align the coordinate systems for the tiles prior to stitching. Typically, each tile is generated by selecting one sequencing cycle and using the selected cycle to define the cluster locations and the coordinate system for the tile. Because the RTA may select a different cycle for each tile, some of the tiles may be misaligned in relation to the other tiles. In these instances, the tile and swath stitching system may select a desired cycle to use to align each of the coordinate systems from the different cycles. Then the tile and swath stitching system may transform the coordinate systems for each of the tiles to the desired cycle, so that the cluster locations in each of the tiles are defined with respect to the same coordinate system.

In accordance with a first implementation, a method for stitching genomic sequence data is described. The method may include: for respective pairs of overlapping swaths of genomic sequence data divided into a plurality of tiles, obtaining, by one or more processors, tile data for respective tiles in the pair of overlapping swaths, wherein the tile data is a dataset corresponding to a region of a sample, the dataset including a coordinate system and a set of reads of genomic sequences at locations within the coordinate system; for respective pairs of adjacent tiles in the pair of overlapping swaths: identifying, by the one or more processors, one or more matching reads at respective locations within the pair of adjacent tiles; determining, by the one or more processors, a pairwise offset for the pair of adjacent tiles based on the respective locations of the one or more matching reads; determining, by the one or more processors, a swath offset for the pair of overlapping swaths based on the pairwise offsets for the respective pairs of adjacent tiles in the pair of overlapping swaths; and/or stitching, by the one or more processors, the respective pairs of adjacent tiles together by aligning the coordinate systems of the respective tiles using the swath offset to generate a continuous coordinate system across the pair of overlapping swaths.

In accordance with the first implementation, the pair of overlapping swaths may include a first swath and a second swath, and stitching respective pairs of adjacent tiles together may include: applying, by the one or more processors, a transformation to the coordinate systems of tiles in the second swath based on the swath offset; determining, by the one or more processors, a middle portion of the continuous coordinate system between the first and second swaths; and/or filtering, by the one or more processors, one or more duplicated reads from the continuous coordinate system, wherein a duplicated read is at a location within the continuous coordinate system on an opposite side of the middle portion from the first or second swath where the duplicated read was identified before the transformation.

In accordance with the first implementation, the plurality of tiles within respective swaths may be overlapping, the pair of overlapping swaths may be adjacent horizontally such that respective pairs of adjacent tiles in the pair of overlapping swaths may be adjacent horizontally, the pairwise offset may be a horizontal pairwise offset, and the swath offset may be a horizontal swath offset. In accordance with this implementation, the method may further include: for respective pairs of vertically adjacent tiles within a particular swath: identifying, by the one or more processors, one or more matching reads at respective locations within the pair of vertically adjacent tiles; determining, by the one or more processors, a vertical pairwise offset for the pair of vertically adjacent tiles based on the respective locations of the one or more matching reads; determining, by the one or more processors, a vertical swath offset for the particular swath based on the vertical pairwise offsets for respective pairs of adjacent tiles in the particular swath; and/or stitching, by the one or more processors, respective pairs of vertically adjacent tiles together by aligning the coordinate systems of respective tiles using the vertical swath offset.

In accordance with the first implementation, the plurality of tiles within respective swaths may be overlapping, the pair of overlapping swaths may be adjacent vertically such that respective pairs of adjacent tiles in the pair of overlapping swaths may be adjacent vertically, the pairwise offset may be a vertical pairwise offset, and the swath offset may be a vertical swath offset. In accordance with this implementation, the method may further include: for respective pairs of horizontally adjacent tiles within a particular swath: identifying, by the one or more processors, one or more matching reads at respective locations within the pair of horizontally adjacent tiles; determining, by the one or more processors, a horizontal pairwise offset for the pair of horizontally adjacent tiles based on the respective locations of the one or more matching reads; determining, by the one or more processors, a horizontal swath offset for the particular swath based on the horizontal pairwise offsets for respective pairs of adjacent tiles in the particular swath; and/or stitching, by the one or more processors, respective pairs of horizontally adjacent tiles together by aligning the coordinate systems of respective tiles using the horizontal swath offset.

In accordance with the first implementation, the method may further comprise: rotating, by the one or more processors, a coordinate system for at least one of the plurality of tiles within the particular swath to align the coordinate system of the tile with coordinate systems for other tiles within the particular swath. In accordance with this implementation, respective tiles may be generated for a particular cycle of a plurality of cycles, and rotating the coordinate system for the tile may include: selecting, by the one or more processors, a desired cycle for use in aligning the coordinate systems of the tiles; and/or aligning, by the one or more processors, the coordinate system of respective tiles to a coordinate system for the desired cycle by applying an affine transformation for the desired cycle. Also in accordance with this implementation, aligning the coordinate system of respective tiles may include: determining, by the one or more processors, one or more of: (i) a translation parameter, (ii) a magnification parameter, or (iii) a shear mapping parameter for the desired cycle; determining, by the one or more processors, a center location of the tile; identifying, by the one or more processors, locations of clusters of reads within the tile; and/or for respective cluster locations, applying, by the one or more processors, one or more of: (i) the translation parameter, (ii) the magnification parameter, or (iii) the shear mapping parameter to a difference between the cluster location and the center location.

In accordance with the first implementation, a genomic sequence at a particular location using the continuous coordinate system may be identified; a genetic variant based on the genomic sequence may be identified; and/or a phenotype associated with the genetic variant may be identified.

In accordance with a second implementation, a system for stitching genomic sequence data is described. The system may include one or more processors; one or more memories coupled to the one or more processors; and/or computer-readable instructions stored in the one or more memories. The computer-readable instructions, when executed by the one or more processors, may cause the system to: for respective pairs of overlapping swaths of genomic sequence data divided into a plurality of tiles, obtain tile data for respective tiles in the pair of overlapping swaths, wherein the tile data is a dataset corresponding to a region of a sample, the dataset including a coordinate system and a set of reads of genomic sequences at locations within the coordinate system, for respective pairs of adjacent tiles in the pair of overlapping swaths: identify one or more matching reads at respective locations within the pair of adjacent tiles, determine a pairwise offset for the pair of adjacent tiles based on the respective locations of the one or more matching reads, determine a swath offset for the pair of overlapping swaths based on the pairwise offsets for the respective pairs of adjacent tiles in the pair of overlapping swaths, and/or stitch respective pairs of adjacent tiles together by aligning the coordinate systems of respective tiles using the swath offset to generate a continuous coordinate system across the pair of overlapping swaths.

In accordance with the second implementation, the pair of overlapping swaths may include a first swath and a second swath, and stitching respective pairs of adjacent tiles together may cause the system to: apply a transformation to the coordinate systems of tiles in the second swath based on the swath offset, determine a middle portion of the continuous coordinate system between the first and second swaths, and/or filter one or more duplicated reads from the continuous coordinate system, wherein a duplicated read is at a location within the continuous coordinate system on an opposite side of the middle portion from the first or second swath where the duplicated read was identified before the transformation.

In accordance with the second implementation, the plurality of tiles within respective swaths may be overlapping, the pair of overlapping swaths may be adjacent horizontally such that respective pairs of adjacent tiles in the pair of overlapping swaths may be adjacent horizontally, the pairwise offset may be a horizontal pairwise offset, and the swath offset may be a horizontal swath offset. In accordance with this implementation, the computer-readable instructions may further cause the system to: for respective pairs of vertically adjacent tiles within a particular swath: identify one or more matching reads at respective locations within the pair of vertically adjacent tiles, determine a vertical pairwise offset for the pair of vertically adjacent tiles based on the respective locations of the one or more matching reads, determine a vertical swath offset for the particular swath based on the vertical pairwise offsets for respective pairs of adjacent tiles in the particular swath, and/or stitch respective pairs of vertically adjacent tiles together by aligning the coordinate systems of respective tiles using the vertical swath offset.

In accordance with the second implementation, the plurality of tiles within respective swaths may be overlapping, the pair of overlapping swaths may be adjacent vertically such that respective pairs of adjacent tiles in the pair of overlapping swaths may be adjacent vertically, the pairwise offset may be a vertical pairwise offset, and the swath offset may be a vertical swath offset. In accordance with this implementation, the computer-readable instructions may further cause the system to: for respective pairs of horizontally adjacent tiles within a particular swath: identify one or more matching reads at respective locations within the pair of horizontally adjacent tiles, determine a horizontal pairwise offset for the pair of horizontally adjacent tiles based on the respective locations of the one or more matching reads, determine a horizontal swath offset for the particular swath based on the horizontal pairwise offsets for respective pairs of adjacent tiles in the particular swath, and/or stitch respective pairs of horizontally adjacent tiles together by aligning the coordinate systems of respective tiles using the horizontal swath offset.

In accordance with the second implementation, the computer-readable instructions may further cause the system to: rotate a coordinate system for at least one of the plurality of tiles within the particular swath to align the coordinate system of the tile with coordinate systems for other tiles within the particular swath. In accordance with this implementation, respective tiles may be generated for a particular cycle of a plurality of cycles, and rotating the coordinate system for the tile may include: select a desired cycle for use in aligning the coordinate systems of the tiles; and/or align the coordinate system of respective tiles to a coordinate system for the desired cycle by applying an affine transformation for the desired cycle. Also in accordance with this implementation, aligning the coordinate system of respective tiles may cause the system to: determine one or more of: (i) a translation parameter, (ii) a magnification parameter, or (iii) a shear mapping parameter for the desired cycle; determine a center location of the tile; identify locations of clusters of reads within the tile; and/or for respective cluster locations, apply one or more of: (i) the translation parameter, (ii) the magnification parameter, or (iii) the shear mapping parameter to a difference between the cluster location and the center location.

In accordance with the second implementation, a genomic sequence at a particular location using the continuous coordinate system may be identified; a genetic variant based on the genomic sequence may be identified; and/or a phenotype associated with the genetic variant may be identified.

In accordance with a third implementation, a non-transitory computer-readable medium storing instructions for stitching genomic sequence data is described. The instructions, when executed by one or more processors, may cause the one or more processors to: for respective pairs of overlapping swaths of genomic sequence data divided into a plurality of tiles, obtain tile data for respective tiles in the pair of overlapping swaths, wherein the tile data is a dataset corresponding to a region of a sample, the dataset including a coordinate system and a set of reads of genomic sequences at locations within the coordinate system, for respective pairs of adjacent tiles in the pair of overlapping swaths: identify one or more matching reads at respective locations within the pair of adjacent tiles, determine a pairwise offset for the pair of adjacent tiles based on the respective locations of the one or more matching reads, determine a swath offset for the pair of overlapping swaths based on the pairwise offsets for the respective pairs of adjacent tiles in the pair of overlapping swaths, and/or stitch the respective pairs of adjacent tiles together by aligning the coordinate systems of respective tiles using the swath offset to generate a continuous coordinate system across the pair of overlapping swaths.

In accordance with the third implementation, the pair of overlapping swaths may include a first swath and a second swath, and stitching respective pairs of adjacent tiles together may cause the one or more processors to: apply a transformation to the coordinate systems of tiles in the second swath based on the swath offset, determine a middle portion of the continuous coordinate system between the first and second swaths, and/or filter one or more duplicated reads from the continuous coordinate system, wherein a duplicated read is at a location within the continuous coordinate system on an opposite side of the middle portion from the first or second swath where the duplicated read was identified before the transformation.

In accordance with the third implementation, the plurality of tiles within respective swaths may be overlapping, the pair of overlapping swaths may be adjacent horizontally such that respective pairs of adjacent tiles in the pair of overlapping swaths may be adjacent horizontally, the pairwise offset may be a horizontal pairwise offset, and the swath offset may be a horizontal swath offset. In accordance with this implementation, the instructions may further cause the one or more processors to: for respective pairs of vertically adjacent tiles within a particular swath: identify one or more matching reads at respective locations within the pair of vertically adjacent tiles, determine a vertical pairwise offset for the pair of vertically adjacent tiles based on the respective locations of the one or more matching reads, determine a vertical swath offset for the particular swath based on the vertical pairwise offsets for respective pairs of adjacent tiles in the particular swath, and/or stitch respective pairs of vertically adjacent tiles together by aligning the coordinate systems of respective tiles using the vertical swath offset.

In accordance with the third implementation, the plurality of tiles within respective swaths may be overlapping, the pair of overlapping swaths may be adjacent vertically such that respective pairs of adjacent tiles in the pair of overlapping swaths may be adjacent vertically, the pairwise offset may be a vertical pairwise offset, and the swath offset may be a vertical swath offset. In accordance with this implementation, the instructions may further cause the one or more processors to: for respective pairs of horizontally adjacent tiles within a particular swath: identify one or more matching reads at respective locations within the pair of horizontally adjacent tiles, determine a horizontal pairwise offset for the pair of horizontally adjacent tiles based on the respective locations of the one or more matching reads, determine a horizontal swath offset for the particular swath based on the horizontal pairwise offsets for respective pairs of adjacent tiles in the particular swath, and/or stitch respective pairs of horizontally adjacent tiles together by aligning the coordinate systems of respective tiles using the horizontal swath offset.

In accordance with the third implementation, the instructions may further cause the one or more processors to: rotate a coordinate system for at least one of the plurality of tiles within the particular swath to align the coordinate system of the tile with coordinate systems for other tiles within the particular swath. In accordance with this implementation, respective tiles may be generated for a particular cycle of a plurality of cycles, and rotating the coordinate system for the tile may include: select a desired cycle for use in aligning the coordinate systems of the tiles; and/or align the coordinate system of respective tiles to a coordinate system for the desired cycle by applying an affine transformation for the desired cycle. Also in accordance with this implementation, aligning the coordinate system of respective tiles may cause the system to: determine one or more of: (i) a translation parameter, (ii) a magnification parameter, or (iii) a shear mapping parameter for the desired cycle; determine a center location of the tile; identify locations of clusters of reads within the tile; and/or for respective cluster locations, apply one or more of: (i) the translation parameter, (ii) the magnification parameter, or (iii) the shear mapping parameter to a difference between the cluster location and the center location.

In accordance with the third implementation, a genomic sequence at a particular location using the continuous coordinate system may be identified; a genetic variant based on the genomic sequence may be identified; and/or a phenotype associated with the genetic variant may be identified.

With regard to performing improved quality control of spatial analysis products/processes, shortcomings of the prior art can be overcome, and benefits as described in this disclosure can be achieved, through systems and devices for improving quality control of substrate analysis products. Various implementations of the systems and devices are described below, and the systems and devices in any combination, may overcome these shortcomings and achieve the benefits described herein.

In one aspect, a computer-implemented method for improving quality control of a spatial transcriptomics substrate includes (i) receiving, via one or more processors, a plurality of digital tile images corresponding to a plurality of sequencing cycles of the spatial transcriptomics substrate; (ii) processing, via one or more processors, each of the digital tile images to determine a set of respective quality control scores; and (iii) generating, via one or more processors, a final quality control score for the spatial transcriptomics substrate, wherein the final quality control score is based on the set of respective quality control scores, and wherein the final quality score predicts how the substrate will perform in a downstream process.

In another aspect, a computing system for improving quality control of a spatial transcriptomics substrate includes one or more processors; and one or more memories having stored thereon computer-executable instructions that, when executed, cause the computing system to: (i) receive a plurality of digital tile images corresponding to a plurality of sequencing cycles of the spatial transcriptomics substrate; (ii) process each of the digital tile images to determine a set of respective quality control scores; and (iii) generate a final quality control score for the spatial transcriptomics substrate, wherein the final quality control score is based on the set of respective quality control scores, and wherein the final quality score predicts how the substrate will perform in a downstream process.

In yet another aspect, a non-transitory computer-readable medium having stored thereon computer-executable instructions that, when executed, cause a computer to: (i) receive a plurality of digital tile images corresponding to a plurality of sequencing cycles of the spatial transcriptomics substrate; (ii) process each of the digital tile images to determine a set of respective quality control scores; and (iii) generate a final quality control score for the spatial transcriptomics substrate, wherein the final quality control score is based on the set of respective quality control scores, and wherein the final quality score predicts how the substrate will perform in a downstream process.

Advantages will become more apparent to those of ordinary skill in the art from the following description of the preferred implementations which have been shown and described by way of illustration. As will be realized, the present implementations may be capable of other and different implementations, and their details are capable of modification in various respects. Accordingly, the drawings and description are to be regarded as illustrative in nature and not as restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The Figures described below depict various implementations of the system and methods disclosed therein. It should be understood that each Figure depicts a particular implementation of the disclosed system and methods, and that each of the Figures is intended to accord with a possible implementation thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present implementations are not limited to the precise arrangements and instrumentalities shown, wherein:

FIG. 1 illustrates a block diagram of an implementation of a computing environment in accordance with the teachings of this disclosure.

FIG. 2 illustrates a block diagram of the components within a genomic sequence stitching system in accordance with the teachings of this disclosure.

FIG. 3 illustrates a flow diagram of an example method for stitching genomic sequence data, which can be implemented in a computing device, such as the one or more server device(s) of FIG. 1.

FIG. 4 illustrates fields of view of adjacent tiles having matching reads at respective locations within the pair of adjacent tiles, which can be used to determine a pairwise offset in accordance with the teachings of this disclosure.

FIG. 5A illustrates a diagram of example overlapping swaths stitched together having duplicated reads in accordance with the teachings of this disclosure.

FIG. 5B illustrates another diagram of the example stitched swaths of FIG. 5A after the duplicated reads have been filtered out in accordance with the teachings of this disclosure.

FIG. 6A illustrates a diagram of example overlapping swaths before stitching in accordance with the teachings of this disclosure.

FIG. 6B illustrates a diagram of the example overlapping swaths of FIG. 6A after stitching in accordance with the teachings of this disclosure.

FIG. 7A illustrates a diagram of example overlapping swaths before a transformation in accordance with the teachings of this disclosure.

FIG. 7B illustrates a diagram of the example overlapping swaths of FIG. 7A after a transformation in accordance with the teachings of this disclosure.

FIG. 8 illustrates a diagram of example workflows of the genomic sequence stitching process in accordance with the teachings of this disclosure.

FIG. 9A depicts an exemplary computing environment for performing quality control for spatial genomics sequencing, according to some aspects.

FIG. 9B depicts an exemplary computer-implemented method of performing spatial barcoding across a high-resolution/large-area substrate, according to some aspects.

FIG. 10A depicts heatmaps showing pass filter counts and transcript counts, according to some aspects.

FIG. 10B depicts heatmaps showing counts/density of 1) decoded PF (pass-filter) barcodes seeded on the substrate during a first sequencing step and 2) captured transcripts during a second sequencing step where tissue sample is placed on the substrate, and correlations between spatial density of barcodes used in the second sequencing step and spatial density of high-quality barcodes decoded in the first sequencing step, according to some aspects.

FIG. 10C depicts sequencing cycle heatmaps, according to some aspects.

FIG. 11A depicts heatmaps showing example artifacts that can be predicted, and correlations between spatial density of barcodes used and spatial density of high-quality barcodes, according to some aspects.

FIG. 11B depicts sequencing cycle heatmaps corresponding to FIG. 11A, according to some aspects.

FIG. 12A depicts heatmaps showing example artifacts that can be predicted, and correlations between spatial density of barcodes used and spatial density of high-quality barcodes for an RNA experiment, according to some aspects.

FIG. 12B depicts sequencing cycle heatmaps corresponding to FIG. 12A, according to some aspects.

FIG. 13A depicts a heatmap of a simulated tile having a uniform distribution, according to some aspects.

FIG. 13B depicts tiles with or without removed areas of different sizes, according to some aspects.

FIG. 14A illustrates heatmaps and histograms illustrating cluster locations and corresponding Poisson distributions, according to some aspects.

FIG. 14B depicts a heatmap of cluster counts and corresponding bin-wise VMR (Variance-to-Mean Ratio), according to some aspects.

FIG. 14C depicts heatmaps of mean density, bin-wise modified VMR, and outlier bins corresponding to FIG. 14B, according to some aspects.

FIG. 14D depicts a histogram illustrating non-uniformity of tile-wise cluster counts across a flow cell, according to some aspects.

FIG. 14E depicts a histogram illustrating non-uniformity of bin-wise cluster counts across a tile, according to some aspects.

FIG. 14F depicts a histogram illustrating VMR with respect to non-uniformities apart from those found in common patterns, according to some aspects.

FIG. 14G depicts a histogram illustrating outlier values of bin-wise VMR_M per tile, according to some aspects.

FIG. 14H depicts a histogram illustrating tiles with outlier VMR_M values, according to some aspects.

FIG. 14I depicts example histograms of tiles with substantial deviations, according to some aspects.

FIG. 15A depicts heatmaps of used barcode counts and high-quality barcode counts, along with a corresponding tissue mask, and correlations of the used barcode counts and the high-quality barcode counts, according to some aspects.

FIG. 15B depicts histograms depicting tissue masks per tile size, high-quality VMR, used count high-quality VMR greater than a threshold, and VMR used greater than the threshold corresponding to FIG. 15A, according to some aspects.

FIG. 15C depicts a chart validating VMR of high-quality VMR with used VMR, according to some aspects.

FIG. 16A depicts tiles with small first VMR (Variance-to-Mean Ratio) and large second VMR, according to some aspects.

FIG. 16B depicts tiles with large first VMR and large second VMR, according to some aspects.

FIG. 16C depicts tiles with small first VMR and small second VMR, according to some aspects.

FIG. 16D depicts a graph corresponding to FIG. 15C with labeled sub-regions corresponding respectively to FIGS. 16A-16C, according to some aspects.

FIG. 16E depicts a graph of correlation of labeled experiments, according to some aspects.

FIG. 16F depicts a scatter plot illustrating minimum thresholds for high specificity of false artifact labels, according to some aspects.

FIG. 17 depicts a computer-implemented method for improving quality control of a spatial transcriptomics substrate, according to some aspects.

The Figures depict preferred implementations for purposes of illustration only. Alternative implementations of the systems and methods illustrated herein may be employed without departing from the principles of the invention described herein.

### DETAILED DESCRIPTION

Although the following text discloses a detailed description of implementations of methods, apparatuses and/or articles of manufacture, it should be understood that the legal scope of the property right is defined by the words of the claims set forth at the end of this document. Accordingly, the following detailed description is to be construed as examples only and does not describe every possible implementation, as describing every possible implementation would be impractical, if not impossible. Numerous alternative implementations could be implemented, using either current technology or technology developed after the filing date of this patent. It is envisioned that such alternative implementations would still fall within the scope of the claims.

The present techniques are directed to methods and systems for quality control of spatial analysis products for spatial analysis sequencing, and more particularly, to methods and systems for improving quality control of spatial transcriptomics substrates, such as those used in conjunction with Illumina sequencing platforms. The present techniques may include computing quality control metrics (e.g., a variety of variance-based statistical measures) with respect to flow cells; for example, for predicting second seq quality from first seq data. Generally, the present techniques may be used to detect non-uniformities (e.g., bubbles or other missing clusters on a substrate). The present techniques are applicable to multiple types of flow cells, including uniform/random flow cells and patterned flow cells. The present techniques may include generating tile-wise, bin-wise, sample-wise, substrate-wise, etc. scores, using predetermined, adjustable threshold values, to control quality of upstream spatial analysis products.

Herein, the term “first seq” generally refers to a first sequencing step in a spatial genomics workflow, wherein barcode sequences on a substrate array are read out together with their spatial coordinates. The term “second seq” generally refers to a second sequencing step in the spatial genomics workflow, wherein the substrate array of the first sequencing step is exposed to a sample whose molecules are captured and read out together with corresponding barcode sequences. “Reading out” the barcode sequences and spatial coordinates may include obtaining and interpreting genetic information (e.g., RNA transcripts) from a biological sample, wherein the obtaining includes preserving the spatial context of where each transcript is located within the biological sample/tissue. Steps involved in “reading out” may include tissue preparation and sequencing by sectioning a tissue sample onto a slide that includes an array of barcoded probes, hybridization and reverse transcription, sequencing using next-generation sequencing, data analysis and spatial mapping by mapping sequencing reads back to locations on the tissue slide using the barcode sequences and interpretation.

Overall, the combination of first seq and second seq processes enable users (e.g., researchers) to create detailed maps of gene expression across different regions of the tissue and to obtain a comprehensive view of the transcriptome in a spatial context. This enables the user to study spatial organization of gene expression at high resolution, for various purposes (e.g., to determine how gene expression varies across different tissue regions, between healthy and diseased states, or during developmental processes).

### Exemplary Computing Environment

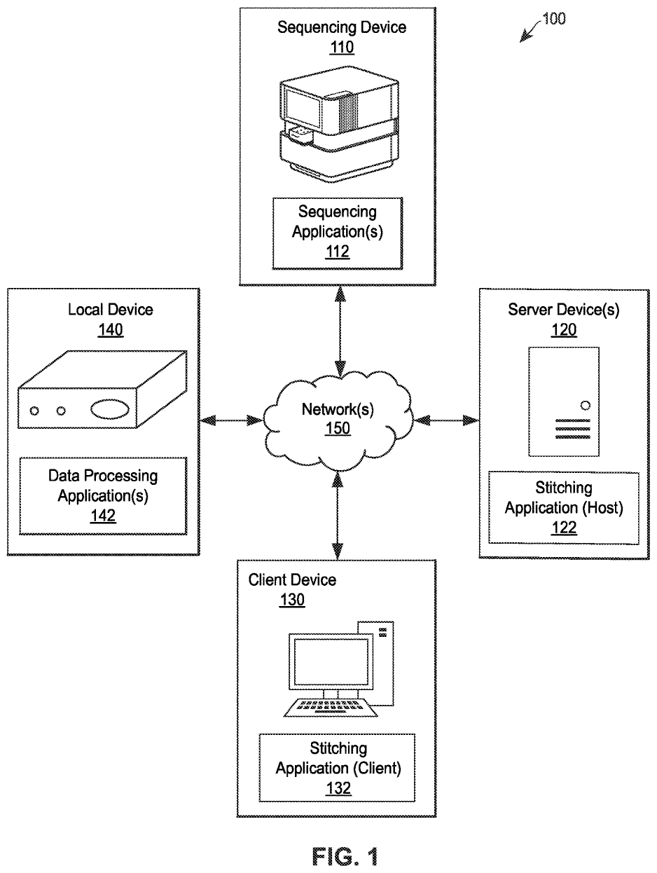

FIG. 1 illustrates a block diagram of an example computing environment 100 which may be used to perform genomic sequencing, perform spatial mapping of genomic sequence data, and/or stitch the spatial map of the genomic sequence data. The example computing environment 100 may include a sequencing device 110, one or more server device(s) 120, a client device 130, and a local device 140.

The sequencing device 110 may comprise a computing device, image sensors, and one or more sequencing applications 112 for sequencing a genomic sample or other nucleic-acid polymer. In some versions, by executing the one or more sequencing applications 112 using a processor, the sequencing device 110 may analyze nucleotide fragments or oligonucleotides extracted from genomic samples to generate nucleotide reads or other data utilizing computer implemented methods and systems either directly or indirectly on the sequencing device 110. More particularly, the sequencing device 110 may receive nucleotide-sample slides (e.g., flow cells) comprising nucleotide fragments extracted from samples and further copies, and the sequencing device may determine the nucleobase sequence of such extracted nucleotide fragments. The sequencing device 110 may be the sequencing system described in U.S. patent application Ser. No. 18/340,795, titled “Split-Read Alignment by Intelligently Identifying and Scoring Candidate Split Groups filed on Jul. 23, 2023, which is hereby incorporated by reference in its entirety.

In some versions, the sequencing device 110 may utilize sequencing-by-synthesis (SBS) to sequence nucleotide fragments into nucleotide reads and determine nucleobase calls for the nucleotide reads. By executing the one or more sequencing applications 112, the sequencing device 110 may further store the nucleobase calls as part of base-call data that is formatted as a binary base call (BCL) file and send the BCL file to the local device 140 and/or the one or more server device(s) 120. The sequencing device 110 may communicate the BCL file and/or other data to the local device 140 and/or the client device 130 via one or more network(s) 150 or directly (e.g., bypassing the one or more network(s) 150).

The sequencing device 110 may image a sample within a flow cell in swaths which are regions of the sample. The flow cell may be a patterned flow cell, having nanowells where DNA clusters form at fixed locations throughout the flow cell, or a non-patterned flow cell (a random flow cell). The swaths may overlap with each other. For example, adjacent swaths may have a 3% overlap.

As used herein, the term “cluster of oligonucleotides” (or simply “cluster(s)” or “DNA cluster(s)”) refers to a localized group or collection of DNA or RNA molecules on a nucleotide-sample slide, such as a flow cell, or other solid surface. In particular, a cluster includes tens, hundreds, thousands, or more copies of a cloned or the same DNA or RNA segment. For example, in one or more embodiments, a cluster includes a grouping of oligonucleotides immobilized in a section of a flow cell or other nucleotide-sample slide. In some embodiments, clusters are evenly spaced or organized in a systematic structure within a patterned flow cell. By contrast, in some cases, clusters are randomly organized within a non-patterned flow cell. A cluster of oligonucleotides can be imaged utilizing one or more light signals. For instance, an oligonucleotide-cluster image may be captured by a camera during a sequencing cycle of light emitted by irradiated fluorescent tags incorporated into oligonucleotides from one or more clusters on a flow cell.

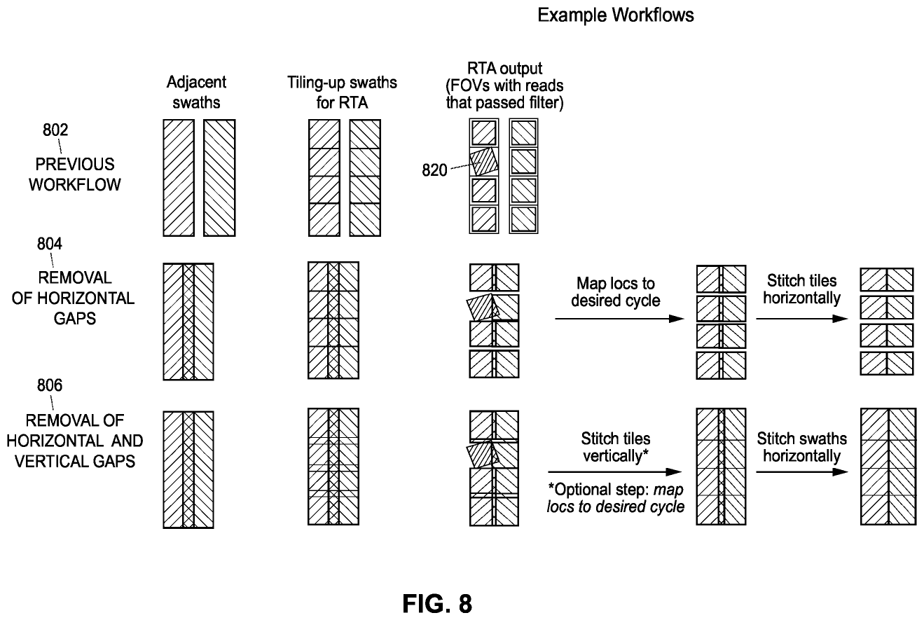

In some scenarios, the local device 140 may be located at or near a same physical location of the sequencing device 110. For instance, the local device 140 and the sequencing device 110 may be integrated into a single computing device. The local device 140 may include and/or execute one or more data processing applications 142 to generate, receive, analyze, store, and/or transmit digital data, such as by generating one or more spatial coordinates for each genomic sequence read and/or correlating one or more spatial coordinates to each corresponding genomic sequence read. By executing software in the form of the one or more data processing applications 142, the local device 140 may determine the spatial coordinates of genomic sequence reads in a sample within a flow cell. More specifically, the local device 140 may divide each swath into tiles (i.e., subregions of the sample), where each tile has its own coordinate system beginning at (0,0). The tiles in one swath may overlap with tiles in another swath since the swaths overlap. Additionally, adjacent tiles within a swath may overlap with each other. For example, the swaths may be images of vertical strips of the flow cell which are adjacent to each other horizontally as shown in FIG. 8. Each swath may be divided vertically into overlapping tiles. In this manner, the local device 140 analyzes the swaths to generate tile data for the tiles within each swath. The tile data may include datasets corresponding to regions of a sample. Each tile represents a subregion and has its own coordinate system with a set of genomic sequence reads at particular locations within the coordinate system. For example, the tile data for one tile may include the read “ATGATGCC” at coordinates (5 μm, 30 μm), the read “GTACGCC” at coordinates (10 μm, 24 μm), etc.

In another implementation, the swaths may be images of horizontal strips of the flow cell which are adjacent to each other vertically. Each swath may be divided horizontally into overlapping tiles. In some implementations, the local device 140 may also send data to the client device 130 and/or the one or more server device(s) 120 (e.g., the tile data indicating genomic reads at particular locations within each tile).

The one or more server device(s) 120 may be located remotely from the local device 140 and/or the sequencing device 110. The one or more server device(s) 120 may comprise a distributed collection of servers, where the one or more server device(s) 120 may include a number of server devices distributed across the one or more network(s) 150 and located in the same or different physical locations. In some implementations, the one or more server device(s) 120 execute or assist in the execution of the one or more data processing applications 142 and/or the one or more sequencing applications 112. The one or more server device(s) 120 may include and/or execute one or more stitching applications 122 to generate, receive, analyze, store, and/or transmit digital data, such as by stitching together one or more sets of genomic sequence data (e.g., tile data). As indicated herein, the one or more server device(s) 120 may, using the one or more stitching applications 122, identify one or more matching genomic sequence reads at respective locations within overlapping tiles and/or swaths and then stitch those tiles and/or swaths together based on the respective locations of the matching reads. The one or more server device(s) 120 may also send data to the client device 130 and/or the local device 140 (e.g., the stitched tiles and/or swaths).

Similar to the one or more server device(s) 120, the client device 130 may be located remotely from the local device 140 and/or the sequencing device 110 and may include one or more stitching applications 132 to generate, receive, analyze, store, and/or transmit digital data, such as by stitching together one or more sets of genomic sequence data. In some implementations, the client device 130 may act as a remote terminal to the one or more server device(s) 120. In these implementations, the one or more server device(s) 120 may execute the one or more stitching applications 122 and the client device 130 may act as a remote input/output device. Additionally or alternatively, in some implementations, the one or more stitching applications 122, 132 may have a host configuration executing on the one or more server device(s) 120 and a client configuration executing on the client device 130, wherein the host configuration has different functions from the client configuration. In these implementations, the two configurations may interact with one other over the one or more network(s) 150. The client device 130 may accordingly present and/or display information pertaining to the stitching of genomic sequence data. For example, the client device 130 may present and/or display graphical representations of the tiles and/or swaths within a graphical user interface of the one or more sequencing applications 112. More specifically, the client device 130 may present a spatial molecular map of a region of tissue without horizontal gaps.

The one or more stitching applications 122, 132 may stitch together adjacent tiles and/or swaths based upon the coordinates of matching reads. For example, as noted above, the dataset may include tiles within particular swaths and the respective locations of reads within a coordinate system for each tile. More specifically, the dataset may include the coordinate systems for Swath 1, Tile 1; Swath 1, Tile 2; . . . Swath 1, Tile n; Swath 2, Tile 1; Swath 2, Tile 2; . . . Swath 2, Tile n; . . . Swath m, Tile 1; Swath m, Tile 2; . . . Swath m, Tile n. Each coordinate system may span a first particular distance (X) in the horizontal direction and a second particular distance (Y) in the vertical direction. Then within each coordinate system, the data indicates reads at particular x, y locations within the coordinate system.

The one or more stitching applications 122, 132 may stitch together the tiles and/or swaths to generate a continuous coordinate system which may span the length and/or width of the flow cell. The one or more server device(s) 120 may perform a base call by identifying a genomic sequence at a particular location using the continuous coordinate system. Additionally, the one or more server device(s) 120 may analyze genomic sequences to identify genetic variants (also referred to herein as “a secondary analysis”). For example, the one or more server device(s) 120 may compare a genomic sequence for the particular location to a reference genome. If the genomic sequence or a portion thereof differs from the reference genome, the one or more server device(s) 120 may identify a genetic variant, such as a substitution, deletion, insertion, copy number variant, duplication, etc. Furthermore, the one or more server device(s) 120 may identify a phenotype associated with the genetic variant (also referred to herein as “a tertiary analysis”), such as an increased risk for a particular disease, an adverse event in response to taking a certain pharmaceutical drug, etc. For example, the one or more server device(s) 120 may obtain a list of phenotypes associated with particular genetic variants from a database.

To stitch the tiles together, the one or more stitching applications 122, 132 may identify matching reads in adjacent tiles (e.g., Swath 1, Tile 1 and Swath 2, Tile 2). Using the coordinates of these matching reads within their respective coordinate systems, the one or more stitching applications 122, 132 may determine a pairwise offset for the pair of tiles. For example, if a matching read in Swath 1, Tile 1 is at (1200 μm, 200 μm) and at (100 μm, 300 μm) in Swath 2, Tile 1, the one or more stitching applications 122, 132 may determine the pairwise offset for Swath 2, Tile 1 is (1100 μm, −100 μm). The stitching application 122, 132 may repeat this process for several matching reads within adjacent tiles and aggregate, average, and/or combine the pairwise offsets for each pair of matching reads in any suitable manner to determine the pairwise offset for the adjacent tiles.

The one or more stitching applications 122, 132 may then stitch together the adjacent tiles by aligning the coordinate systems of the adjacent tiles using the pairwise offset. Then the one or more stitching applications 122, 132 may filter out duplicate reads from the continuous coordinate system across the pair of tiles and/or swaths.

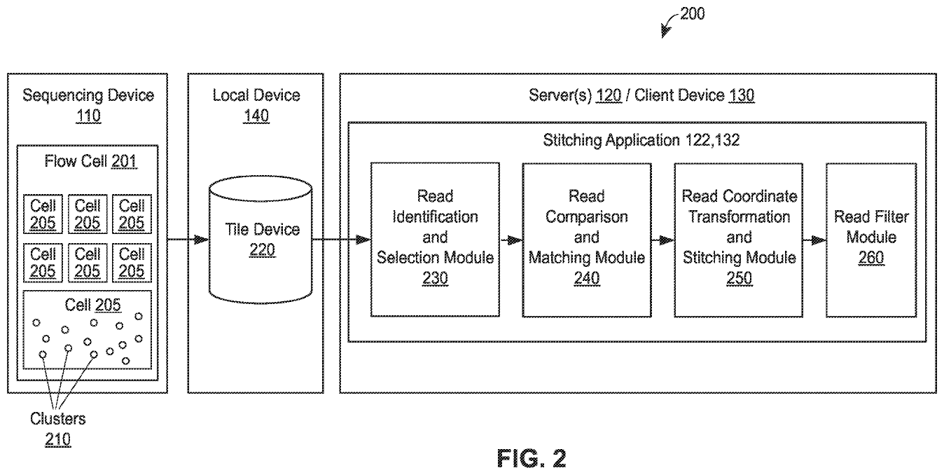

FIG. 2 illustrates an example implementation 200 of the one or more stitching applications 122, 132. The sequencing device 110 may include a flow cell 201, which, in turn, may include a plurality of cells 205. As noted above, the one or more sequencing applications 112 of the sequencing device 110 may identify sequences from DNA clusters 210, also referred to as reads. The sequencing device 110 may output the resulting read data to the local device 140 which in turn may generate spatial coordinates of each of the reads in the tile data 220 (e.g., using the one or more data processing applications 142 as described above). The tile data 220 may then be transmitted to the one or more server device(s) 120 and/or the client device 130.

In the example implementation 200 of the one or more stitching applications 122, 132, the tile data 220 may be input into a read identification and selection module 230, a read comparison and matching module 240, a read coordinate transformation and stitching module 250, and a read filter module 260. The foregoing modules may be separate and/or distinct processors and/or more memory units (e.g., dedicated micro-processors), general purpose processor(s) executing one or more application(s) stored on memory, and/or any combination thereof.

The read identification and selection module 230 may receive the tile data 220 from the local device 140 and may search for matching reads in adjacent tiles and/or swaths.

Once the read identification and selection module 230 has identified matching reads in the adjacent tiles and/or swaths, the read identification and selection module 230 may pass the identified matching reads to the read comparison and matching module 240. In some implementations, the read comparison and matching module 240 may compare the difference between the respective locations of a matching read to a maximum threshold offset. The maximum threshold offset may be based on the distance between the respective locations of a matching read within their respective coordinate systems. For example, if a matching read in Swath 1, Tile 1 is in the upper right portion of its coordinate system, and the matching read in Swath 2, Tile 1 is in the upper left portion of its coordinate system, the difference between the respective locations will not exceed the maximum threshold offset. However, if the matching read in Swath 1, Tile 1 is in the lower left portion of its coordinate system, and the matching read in Swath 2, Tile 1 is in the upper right portion of its coordinate system, the difference between the respective locations will exceed the maximum threshold offset.

If the matching reads are at locations which are further apart than the maximum threshold offset, the read comparison and matching module 240 may discard the matching read from the set of matching reads between the adjacent tiles. In this scenario, the matching read is unlikely to correspond to the same read within overlapping portions of adjacent tiles. Instead, the matching read is more likely to include the same sequence in different regions of a sample. Thus, in this instance, the respective locations of the matching read are not useful for determining the offset between the tiles. Accordingly, the matching read is discarded.

The read comparison and matching module 240 may then pass the remaining set of matching reads to the read coordinate transformation and stitching module 250. The read coordinate transformation and stitching module 250 may then determine a pairwise offset between matching reads based on the difference in the coordinates of the read in a first tile and/or swath and the coordinates of the read in a second tile and/or swath. For example, if a matching read in Swath 1, Tile 1 is at (1200 μm, 200 μm) and at (100 μm, 300 μm) in Swath 2, Tile 1, the read comparison and matching module 240 may determine the pairwise offset for Swath 2, Tile 1 is (1100 μm, −100 μm). The read coordinate transformation and stitching module 250 may repeat this process for several matching reads within the first and second tiles and aggregate, average, and/or combine the pairwise offsets for each pair of matching reads in any suitable manner to determine the pairwise offset for the first and second tiles.

The read coordinate transformation and stitching module 250 may use the pairwise offset to align the coordinate systems of the first and second tiles. For example, if Swath 1, Tile 1 spans from coordinates (0, 0) to (1500 μm, 1500 μm), Swath 2, Tile 1 spans from coordinates (0, 0) to (1500 μm, 1500 μm) and the pairwise offset for the adjacent tiles is (1100 μm, −100 μm), then the coordinate system for Swath 2, Tile 1 may be aligned to span from coordinates (1100 μm, −100 μm) to (2600 μm, 1400 μm). In this manner, the coordinate systems for Swath 1, Tile 1 and Swath 2, Tile 1 are combined to generate a continuous coordinate system spanning from coordinates (0, 0) to (2600 μm, 1400 μm).

In some implementations, the read coordinate transformation and stitching module 250 may repeat this process for each pair of adjacent tiles to align their coordinate systems. In this manner, the coordinate systems are aligned across all pairs of adjacent tiles and/or swaths in a flow cell to generate a continuous coordinate system across the flow cell.

In other implementations, the read coordinate transformation and stitching module 250 may determine a pairwise offset for each pair of adjacent tiles within a pair of overlapping swaths. Then the read coordinate transformation and stitching module 250 may determine a swath offset for the pair of overlapping swaths by aggregating, averaging, and/or combining the pairwise offsets in any suitable manner. The read coordinate transformation and stitching module 250 may then apply the swath offset to each of tiles within a swath to align the coordinate systems of the tiles within the overlapping swaths. For example, instead of determining a different pairwise offset for each pair of adjacent tiles and then aligning the adjacent tiles using the pairwise offset, the read coordinate transformation and stitching module 250 may determine one swath offset to use when aligning each pair of adjacent tiles within a pair of overlapping swaths.

The tiles with aligned coordinate systems may be referred to herein as “stitched tiles.” The read coordinate transformation and stitching module 250 may then pass the stitched tiles and/or swaths to the read filter module 260. The read filter module 260 may then filter out any duplicate reads in the stitched tile and/or swath (e.g., matching reads with two instances of the same read after the tiles have been stitched together).

To filter out duplicate reads in a stitched tile that includes a pair of adjacent tiles in first and second swaths, the read filter module 260 may determine a midpoint or middle portion within the continuous coordinate system between the first and second swaths. The midpoint may be the halfway point within the continuous coordinate system for the pair of adjacent tiles. For example, the continuous coordinate system may span from 3000 to 3600 μm in the direction of the adjacent tiles, and the midpoint may be at 3300 μm. In other implementations, the midpoint may be a location at the halfway point within an overlapping region in the adjacent tiles. For example, the overlapping region may span from 3400 to 3500 μm in the direction of the adjacent tiles, and the midpoint may be at 3450 μm.

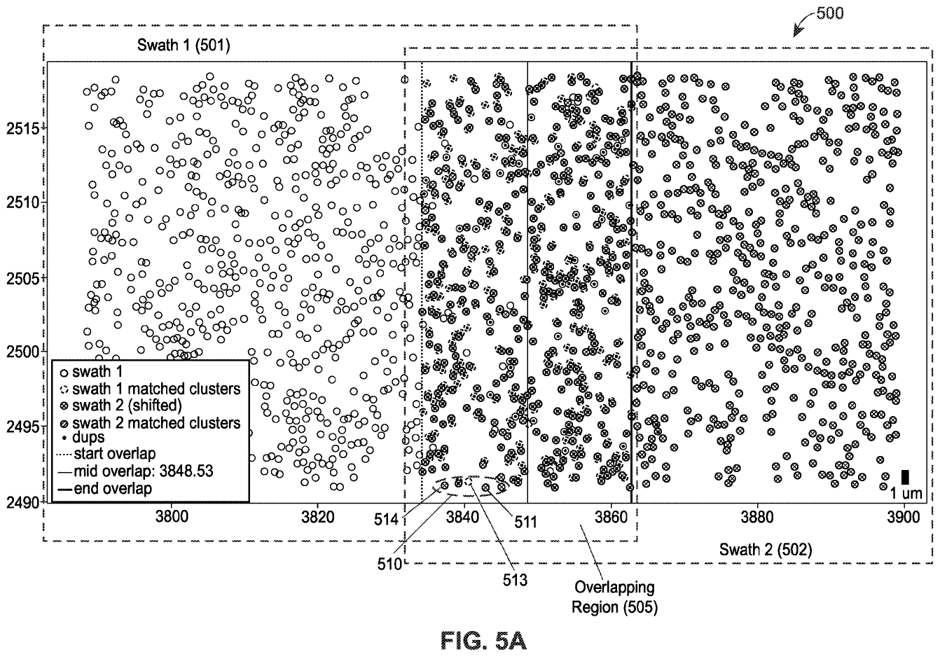

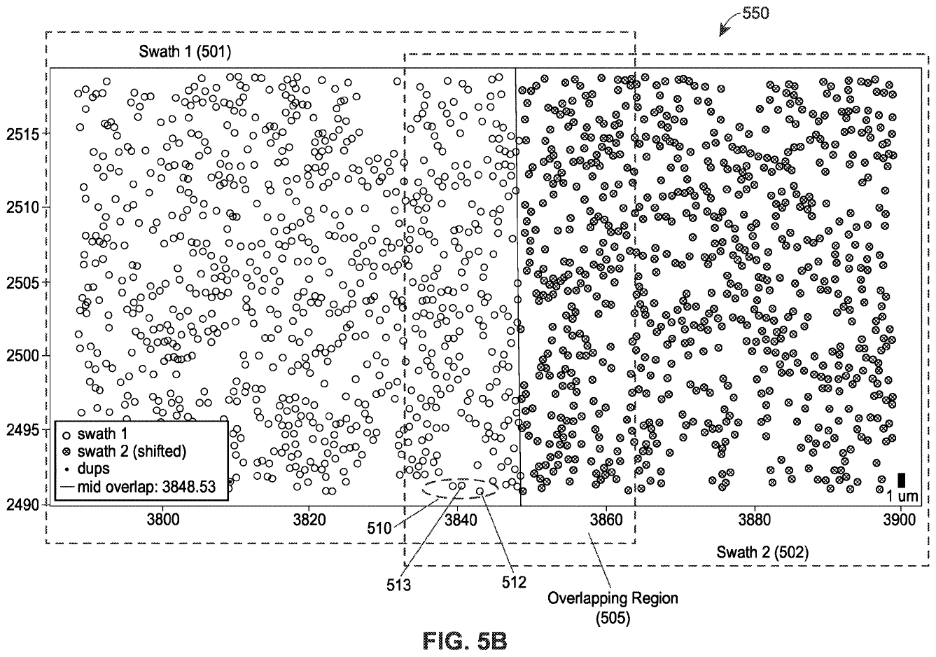

Then the read filter module 260 may filter out duplicate reads on the opposite side of the midpoint or middle portion from the tile and/or swath where the duplicate read was identified before the transformation. For example, if Swath 1, Tile 1 is to the left of Swath 2, Tile 1 in the flow cell, and a duplicate read between the tiles is to the left of the midpoint, then the read filter module 260 may filter out the instance of the duplicate read from Swath 2, Tile 1 and retain the instance of the duplicate read from Swath 1, Tile 1. In another example, if Swath 1, Tile 1 is above Swath 1, Tile 2 in the flow cell, and a duplicate read between the tiles is below the midpoint, then the read filter module 260 may filter out the instance of the duplicate read from Swath 1, Tile 1 and retain the instance of the duplicate read from Swath 1, Tile 2. This is discussed in more detail below with reference to FIGS. 5A and 5B.

### Exemplary Method

The devices and systems described herein may include one or more computers that may be configured to perform particular operations or actions by virtue of having software, firmware, hardware, or a combination of them installed on the system that in operation causes or cause the system to perform the actions, e.g., stitching genomic sequence data.

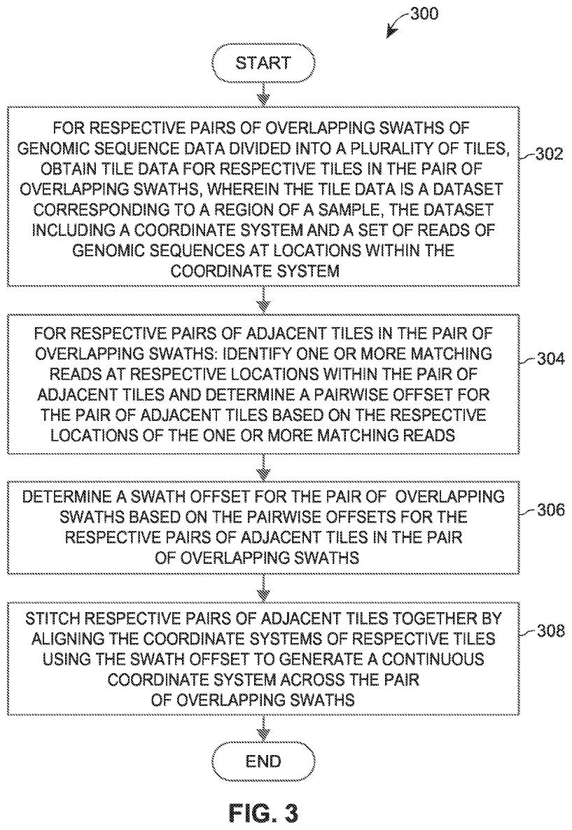

As an illustrative example, FIG. 3 depicts a flow chart for a method 300 using the devices and/or systems described herein.

For respective pairs of overlapping swaths of genomic sequence data divided into tiles, the method 300 may begin by obtaining tile data for respective tiles in the pair of overlapping swaths (block 302). The tile data is a dataset corresponding to a region of a sample. The dataset may include a coordinate system and a set of reads of genomic sequences at locations within the coordinate system.

As described herein, the swaths may be generated by sequencing tissue samples via a flow cell (e.g., the flow cell 201), a sequencing device (e.g., the sequencing device 110), and/or any number of other devices (e.g., the local device 140, the one or more server device(s) 120, the client device 130, etc.). Then the swaths may be divided into tiles and analyzed (e.g., using RTA) to generate dataset(s) indicating the swath, the tile, and genomic reads at respective locations within a coordinate system for the tile. The resulting dataset may include both the genomic sequence and the spatial coordinates of each of the reads. In many implementations, this process is performed in several long passes across the flow cell, herein referred to as swaths. These swaths may then be divided into tiles for faster processing (e.g., by processing several tiles at once in parallel).

In some implementations, the swaths and the tiles have regular dimensions (e.g., each swath has a width of about 1095 microns and each tile has a width of about 1095 microns and a length of about 1095 microns). In some implementations, the swaths are vertically oriented. In other implementations, the swaths are horizontally oriented. Further, as noted above, the swaths and/or the tiles may overlap.

For respective pairs of adjacent tiles in the pair of overlapping swaths, the method 300 may include identifying one or more matching reads at respective locations within the pair of adjacent tiles, and/or determining a pairwise offset for the pair of adjacent tiles based on the respective locations of the one or more matching reads (block 304).

As noted above, adjacent swaths and/or adjacent tiles within swaths may be overlapping (e.g., by 3%). As such, matching reads may be identified in adjacent swaths/tiles by comparing the reads.

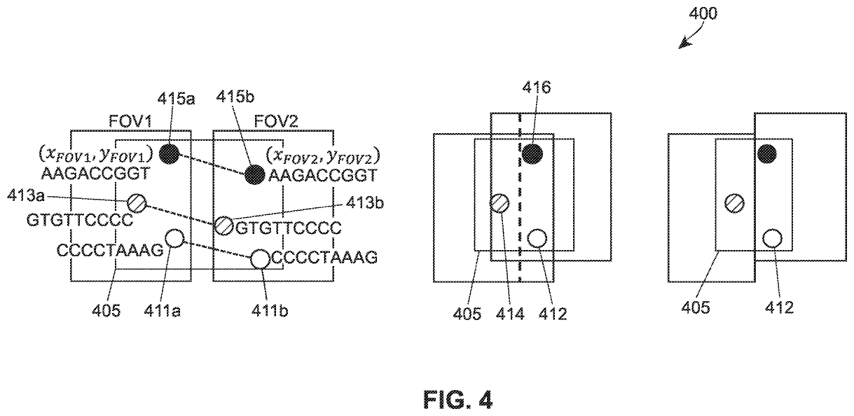

To determine a pairwise offset of adjacent tiles, a computing device (e.g., the one or more server device(s) 120) identifies matching reads each having the same sequence at respective locations within the adjacent tiles. The computing device then uses the respective locations of the matching read as a reference point by which to translate the coordinates of one of the adjacent tiles and/or swaths and stitch the adjacent tiles and/or swaths together (e.g., as demonstrated below with reference to FIGS. 4-6B). However, in some instances a matching read may be identified which is not the same read within overlapping portions of adjacent tiles. In these instances, the matching read may include the same sequence in different regions of a sample. To distinguish between a matching read within overlapping portions of adjacent tiles and a matching read that includes the same sequence in different regions of a sample, the computing device may compare the difference between the respective locations of a matching read to a maximum threshold offset. The maximum threshold offset may be based on the distance between the respective locations of a matching read within their respective coordinate systems.

If the matching reads are at locations which are further apart than the maximum threshold offset, the computing device may discard the matching read from the set of matching reads between the adjacent tiles.

The method 300 may continue by determining a swath offset for the pair of overlapping swaths based on the pairwise offsets for the respective pairs of adjacent tiles in the pair of overlapping swaths (block 306).

For example, if tile 2 of swath 2 is adjusted by an offset of 40 microns in the vertical y direction and −20 microns in the horizontal x direction, tile 3 of swath 2 is adjusted by an offset of 34 microns in the vertical y direction and 10 microns in the x direction, etc., a swath offset for swath 2 may be calculated by aggregating, averaging, and/or combining the pairwise offsets in any suitable manner.

The method 300 may continue by stitching respective pairs of adjacent tiles together by aligning the coordinate systems of respective tiles using the swath offset to generate a continuous coordinate system across the pair of overlapping swaths (block 308).

In some implementations, the tile and swath stitching system may use the swath offset for adjacent swaths to stitch the swaths together. In some implementations, swaths may be stitched without the use of swath offsets. In these implementations, tiles between adjacent swaths may be stitched together using the pairwise offsets for each pair of adjacent tiles.

### Exemplary Implementations

Details of operation of the methods and systems described herein are provided with respect to FIGS. 4-7C.

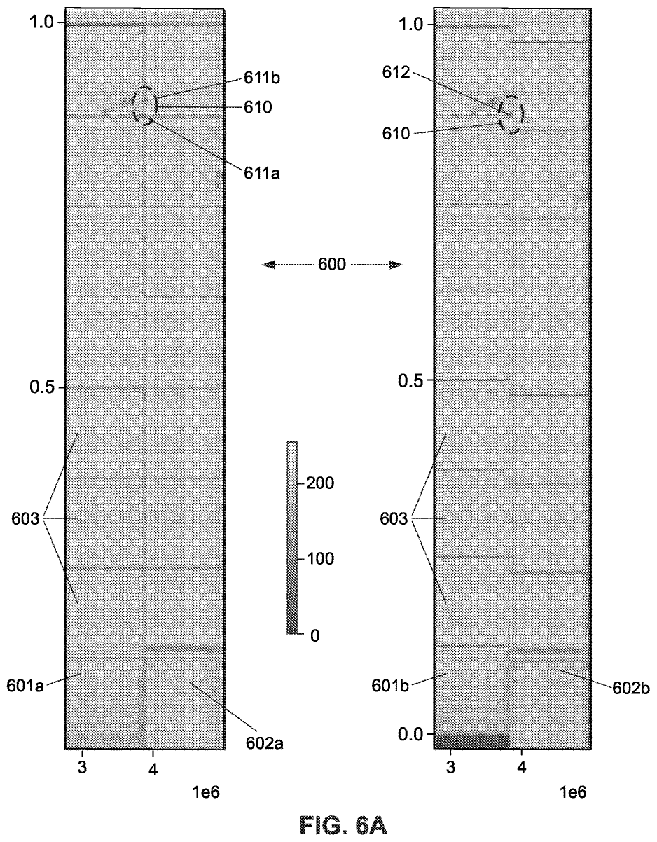

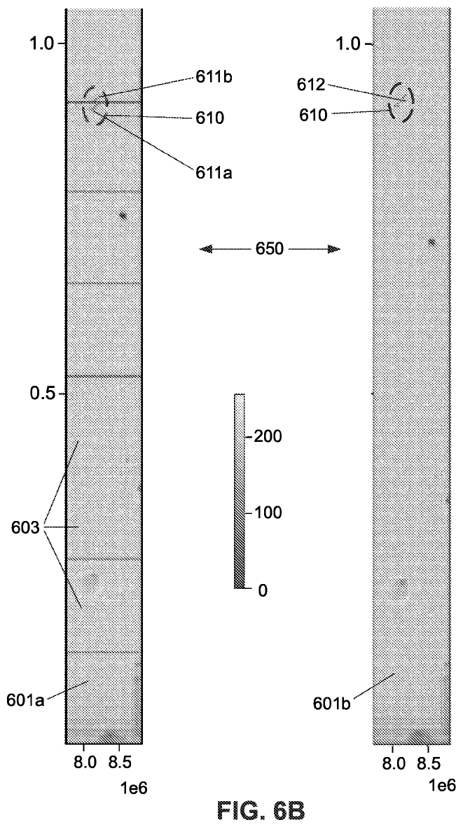

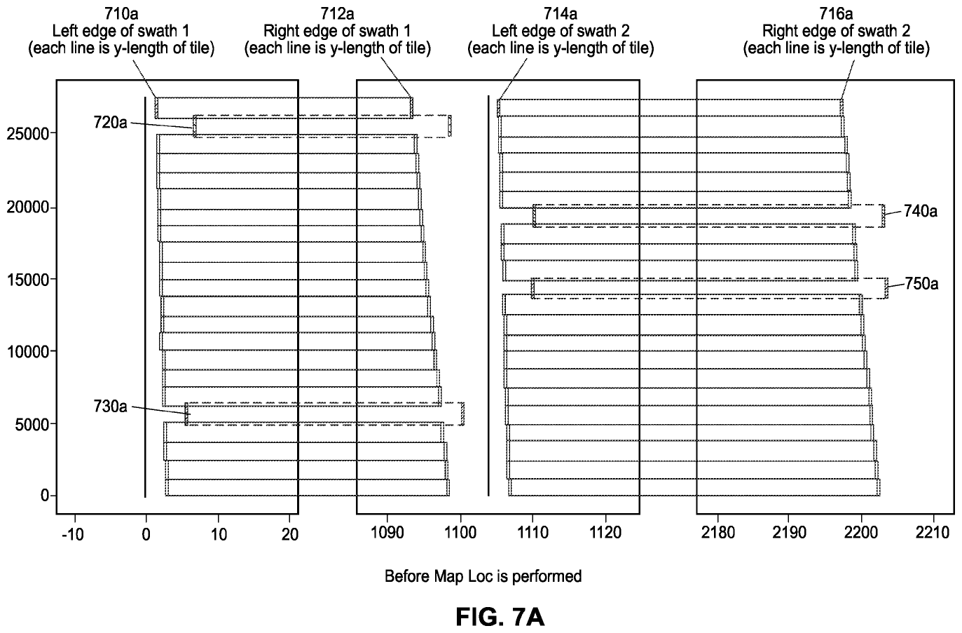

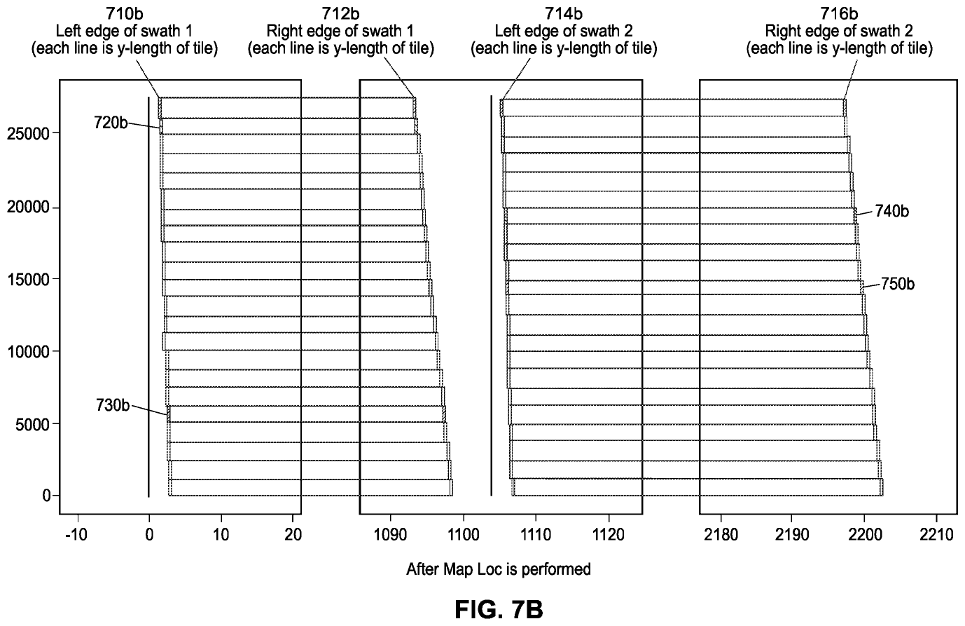

Particularly, FIG. 4 depicts fields of view of adjacent tiles having matching reads at respective locations within the pair of adjacent tiles. FIGS. 5A-5B depict example overlapping swaths which are stitched together. FIGS. 6A-6B depict another example of overlapping swaths which are stitched together. FIGS. 7A-7C depict example overlapping swaths having coordinate systems which are mapped to a desired cycle of several cycles (referred to as a “map loc”).

As illustrated in FIG. 4, fields of view of adjacent tiles (FOV₁, FOV₂) include an overlapping region 405 that may lie between the adjacent tiles and/or swaths. The overlapping region 405 may include reads 411a-415b across the adjacent tiles and/or swaths. For example, a first tile and/or swath (e.g., the left tile and/or swath as illustrated in FIG. 4) may have a first set of matching reads 411a that correspond to a second set of matching reads 411b in the second tile and/or swath (e.g., the right tile and/or swath as illustrated in FIG. 4).

A computing device, such as the one or more server device(s) 120, may identify a first matching read 411a, 411b by identifying identical sequences in each tile (“CCCCTAAAG”). Then the one or more server device(s) 120 may determine a first pairwise offset for the first matching read based on the locations of the first matching read 411a, 411b within the fields of view. The one or more server device(s) 120 may also identify a second matching read 413a, 413b by identifying identical sequences in each tile (“GTGTTCCCC”). Then the one or more server device(s) 120 may determine a second pairwise offset for the second matching read based on the locations of the second matching read 413a, 413b within the fields of view. Additionally, the one or more server device(s) 120 may identify a third matching read 415a, 415b by identifying identical sequences in each tile (“AAGACCGGT”). Then the one or more server device(s) 120 may determine a third pairwise offset for the third matching read based on the locations (x_{FOV1}, y_{FOV1}) and (x_{FOV2}, y_{FOV2}) of the third matching read 415a, 415b within the fields of view. The one or more server device(s) 120 may combine the pairwise offsets for the matching reads in any suitable manner to determine a pairwise offset for the adjacent tiles. For example, the one or more server device(s) 120 may aggregate, average, and/or combine the first, second, and third pairwise offsets for the first, second, and third matching reads to determine the pairwise offset for the adjacent tiles.

The one or more server device(s) 120 may then stitch the adjacent tiles and/or swaths together according to the determined pairwise offset for the adjacent tiles and filter out any duplicate reads. For example, the resulting stitched tile and/or swath may only have filtered reads 412-416 in the overlapping region 405.

The process for filtering duplicate reads is described with reference to FIGS. 5A-5B. As illustrated in FIG. 5A, a first swath 501 is stitched to an adjacent second swath 502. The first swath 501 and the second swath 502 may have an overlapping region 505 (e.g., with a 3% overlap) that may contain several reads 510. The reads 510 in the overlapping region 405 may including duplicate reads 511 between the adjacent swaths, non-duplicate reads 513 found only in the first swath 501, and non-duplicate reads 514 found only in the second swath 502.

FIG. 5B illustrates the stitched first and second swaths 501, 502 after duplicate reads have been filtered out. For example, as mentioned above, the one or more server device(s) 120 may determine a midpoint or middle portion (e.g., at 3848.53 μm) within the continuous coordinate system between the first and second swaths 501, 502. Then the one or more server device(s) 120 may filter out duplicate reads on the opposite side of the midpoint or middle portion from the tile and/or swath where the duplicate read was identified before the transformation. For example, the one or more server device(s) 120 may filter out duplicate reads from the first swath 501 to the right of the midpoint and duplicate reads from the second swath 502 to the left of the midpoint. As shown in FIG. 5B, the remaining reads to the left of the midpoint are from the first swath 501, and the remaining reads to the right of the midpoint are from the second swath 502.

FIGS. 6A and 6B illustrate the two swaths which are stitched together. As illustrated, the left side of FIG. 6A depicts a first swath 601a and a second swath 602a before stitching and the right side of FIG. 6A depicts the first swath 601b and the second swath 602b after stitching. Similarly, as illustrated, the left side of FIG. 6B depicts a plurality of adjacent tiles 603 of a first swath 601a before stitching and the right side of FIG. 6B depicts the plurality of adjacent tiles 603 of the first swath 601b after stitching.

Referring to FIG. 6A, a first swath 601a and an adjacent second swath 602a may be initially unstitched, and both the first swath 601a and the second swath 602a may be divided into adjacent tiles 603. A number of reads 610 may be in an overlapping region between the first swath 601a and the second swath 602a. the one or more server device(s) 120 may identify a matching read 611a, 611b in adjacent tiles within the adjacent swaths 601a, 602a. Then the one or more server device(s) 120 may determine a pairwise offset for the matching read 611a, 611b based on the locations of the matching read 611a, 611b within the adjacent tiles. the one or more server device(s) 120 may combine the pairwise offsets for the matching reads in any suitable manner to determine a pairwise offset for the adjacent tiles. the one or more server device(s) 120 may then stitch the adjacent tiles and/or swaths 601a, 602a together according to the determined pairwise offset for the adjacent tiles and filter out any duplicate reads.

As shown on the right side of FIG. 6A, the first and second swaths 601b, 602b are stitched together for example, using pairwise offsets between adjacent tiles and/or a swath offset between the pair of first and second swaths 601b, 602b. Duplicate reads are filtered out resulting in only filtered reads 612 being included.

Referring to FIG. 6B, adjacent tiles 603 within a first swath 601a may be initially unstitched, as illustrated by the left swath 601a. A number of reads 610 may be in an overlapping region between adjacent tiles.

The one or more server device(s) 120 may identify a matching read 611a, 611b in adjacent tiles within the adjacent swaths 601a, 602a. Then the one or more server device(s) 120 may determine a pairwise offset for the matching read 611a, 611b based on the locations of the matching read 611a, 611b within the adjacent tiles. The one or more server device(s) 120 may combine the pairwise offsets for the matching reads in any suitable manner to determine a pairwise offset for the adjacent tiles. The one or more server device(s) 120 may then stitch the adjacent tiles and/or swaths 601a, 602a together according to the determined pairwise offset for the adjacent tiles and filter out any duplicate reads.

In alternative sequencing analysis systems, tiles may be unintentionally rotated relative to each other during RTA. This is typically due to selecting different sequencing cycles on a tile-by-tile basis when generating the coordinates for each tile. This tile rotation is illustrated in FIG. 8, tile 820 and FIG. 7A.

FIG. 7A depicts two adjacent swaths 710a-716a. Some of the tiles within the respective swaths (e.g., tiles 720a, 730a in swath 1, and tiles 740a, 750a in swath 2) are misaligned in the horizontal x-direction when compared to the coordinates of the other tiles in their respective swaths (e.g., in this illustrative example, by about 5 microns). To address this issue, the one or more server device(s) 120 may transform the coordinate systems of tiles using location mapping (also referred to herein as “map loc”), where a particular sequencing cycle is selected across all tiles of a swath and the coordinate systems of the respective tiles are aligned to the coordinate system for the desired cycle by applying an affine transformation for the desired cycle.

In this manner, the one or more server device(s) 120 may revert the affine transformation applied in RTA so that each of the coordinate systems are defined with respect to the same cycle. In some implementations, the one or more server device(s) 120 may revert the affine transformation applied in RTA for a tile and then apply the affine transformation for the desired cycle to the tile to align the coordinate system for the desired cycles.

More specifically, the one or more server device(s) 120 may map coordinates of the tiles to the coordinates with respect to a desired cycle, using:

[cycleₓ cycle_{y}]=[locsₓ−centerₓ locs_{y}−center_{y}]×A+[Tₓ T_{y}]+[centerₓ center_{y}] (Equation 1)

where:

$$A = [ \begin{matrix}{M}_{x} + 1 & {S}_{xy} \\ {S}_{yx} & {M}_{y} + 1\end{matrix} ] \qquad ( Equation 2 )$$

T, M, S are translation, magnification and shear affine parameters for the desired cycle,
centerₓ, center_{y} are defined through SubRegionSize and RoiRadiusInPixels in RTA's config params, and
locsₓ, locs_{y} are the cluster locations.

This vertically aligns the tiles even when there are no vertical overlaps, so that locations across the whole swath are defined with respect to the same coordinate system.

For each tile, the one or more server device(s) 120 may determine (i) a translation parameter, (ii) a magnification parameter, and/or (iii) a shear mapping parameter for the desired cycle. The one or more server device(s) 120 may also determine the center location of the respective tile and identifies cluster locations within the respective tile. Then for each cluster location, the one or more server device(s) 120 may apply (i) the translation parameter, (ii) the magnification parameter, and/or (iii) the shear mapping parameter to the difference between the respective cluster location and the center location to rotate the coordinate system for the tile so that each of the tiles are aligned.

FIG. 7B depicts the result of the location mapping on the adjacent swaths 710b-716b. Notably, the previously misaligned tiles 720b-750b are no longer misaligned after a map loc is performed.

### Exemplary Workflows

FIG. 8 depicts a diagram of example workflows of the genomic sequence stitching process described herein. The top row of FIG. 8 details the previous workflow 802 where adjacent swaths are non-overlapping with a horizontal gap (e.g., a 100 μm gap) between neighboring swaths. Additionally, in the previous workflow 802, the coordinate systems of tiles are misaligned after RTA. For example, tile 820 is misaligned relative to the other tiles within the swath.

The middle row of FIG. 8 details a workflow 804 for aligning tiles in overlapping swaths. As illustrated, swaths of genomic sequence data are generated and are divided into tiles for processing. Adjacent swaths overlap, but in this illustrative example adjacent tiles within each of the swaths do not overlap. After the RTA analysis, the coordinate systems of the tiles are misaligned. Accordingly, the one or more server device(s) 120 may transform the coordinate systems of tiles using location mapping to align the coordinate systems. Then the one or more server device(s) 120 may stitch the tiles horizontally using the methods described herein.

However, as shown in the workflow 804, the RTA may create a vertical gap between the tiles (e.g., about 10 μm) even though the swaths were divided into contiguous tiles. To remove the vertical gaps, the one or more server device(s) 120 may divide each swath into overlapping tiles (e.g., having about a 3% overlap).

The bottom row of FIG. 8 details a workflow 806 for aligning tiles in overlapping swaths, where each swath is divided into overlapping tiles. After the RTA analysis, the coordinate systems of the tiles are misaligned. Accordingly, the one or more server device(s) 120 may transform the coordinate systems of tiles using location mapping to align the coordinate systems. Then the one or more server device(s) 120 may stitch the tiles vertically using the methods described herein. Next, the one or more server device(s) 120 may stitch the tiles horizontally using the methods described herein. In some implementations, in the workflow 806, the one or more server device(s) 120 may not transform the coordinate systems of tiles using location mapping and instead may proceed to stitch the tiles vertically.

Additionally, while the workflows 804, 806 illustrates the swaths as vertical strips which are adjacent horizontally, the swaths may be horizontal strips which are adjacent vertically. In this implementation, the swaths are divided into horizontally overlapping tiles, and the tiles are stitched horizontally and then vertically.

Example 1. A method for stitching genomic sequence data, the method comprising: obtaining, by one or more processors, tile data for respective tiles in a pair of overlapping swaths of genomic sequence data, wherein the tile data is a dataset corresponding to a region of a sample, the dataset including a coordinate system and a set of reads of genomic sequences at locations within the coordinate system; for respective pairs of adjacent tiles in the pair of overlapping swaths: (i) identifying, by the one or more processors, one or more matching reads at respective locations within the pair of adjacent tiles; and (ii) determining, by the one or more processors, a pairwise offset for the pair of adjacent tiles based on the respective locations of the one or more matching reads; determining, by the one or more processors, a swath offset for the pair of overlapping swaths based on the pairwise offsets for the respective pairs of adjacent tiles in the pair of overlapping swaths; and stitching, by the one or more processors, the respective pairs of adjacent tiles together by aligning the coordinate systems of the respective tiles using the swath offset to generate a continuous coordinate system across the pair of overlapping swaths.

Example 2. The method of example 1, wherein the pair of overlapping swaths includes a first swath and a second swath, and wherein stitching respective pairs of adjacent tiles together includes: applying, by the one or more processors, a transformation to the coordinate systems of tiles in the second swath based on the swath offset; determining, by the one or more processors, a middle portion of the continuous coordinate system between the first and second swaths; and filtering, by the one or more processors, one or more duplicated reads from the continuous coordinate system, wherein a duplicated read is at a location within the continuous coordinate system on an opposite side of the middle portion from the first or second swath where the duplicated read was identified before the transformation.

Example 3. The method of example 1 or example 2, wherein the respective tiles within the pair of swaths are overlapping, wherein the pair of overlapping swaths are adjacent horizontally such that respective pairs of adjacent tiles in the pair of overlapping swaths are adjacent horizontally, the pairwise offset is a horizontal pairwise offset, and the swath offset is a horizontal swath offset, and further comprising: for respective pairs of vertically adjacent tiles within a particular swath: (i) identifying, by the one or more processors, one or more matching reads at respective locations within the pair of vertically adjacent tiles; and (ii) determining, by the one or more processors, a vertical pairwise offset for the pair of vertically adjacent tiles based on the respective locations of the one or more matching reads; determining, by the one or more processors, a vertical swath offset for the particular swath based on the vertical pairwise offsets for the respective pairs of vertically adjacent tiles in the particular swath; and stitching, by the one or more processors, respective pairs of vertically adjacent tiles together by aligning the coordinate systems of respective tiles using the vertical swath offset.

Example 4. The method of any of the preceding examples, further comprising: rotating, by the one or more processors, a coordinate system for at least one of the plurality of tiles within the particular swath to align the coordinate system of the tile with coordinate systems for other tiles within the particular swath.

Example 5. The method of any of the preceding examples, wherein respective tiles are generated for a particular cycle of a plurality of cycles, and wherein rotating the coordinate system for a respective tile includes: selecting, by the one or more processors, a desired cycle for use in aligning the coordinate systems of the respective tiles; and aligning, by the one or more processors, the coordinate systems of the respective tiles to a coordinate system for the desired cycle by applying an affine transformation for the desired cycle.

Example 6. The method of any of the preceding examples, wherein aligning the coordinate systems of the respective tiles includes: for the respective tiles: determining, by the one or more processors, for the desired cycle, one or more of: (i) a translation parameter, (ii) a magnification parameter, or (iii) a shear mapping parameter; determining, by the one or more processors, a center location of the respective tile; identifying, by the one or more processors, locations of clusters of reads within the respective tile; and for respective cluster locations, applying, by the one or more processors, to a difference between the respective cluster location and the center location, one or more of: (i) the translation parameter, (ii) the magnification parameter, or (iii) the shear mapping parameter.

Example 7. The method of any of the preceding examples, further comprising at least one of: identifying, by the one or more processors, a genomic sequence at a particular location using the continuous coordinate system; identifying, by the one or more processors, a genetic variant based on the genomic sequence; or identifying, by the one or more processors, a phenotype associated with the genetic variant.

Example 8. A system for stitching genomic sequence data, the system comprising: one or more processors; one or more memories coupled to the one or more processors; and computer-readable instructions stored in the one or more memories that, when executed by the one or more processors, cause the system to: obtain tile data for respective tiles in a pair of overlapping swaths of genomic sequence data, wherein the tile data is a dataset corresponding to a region of a sample, the dataset including a coordinate system and a set of reads of genomic sequences at locations within the coordinate system, for respective pairs of adjacent tiles in the pair of overlapping swaths: (i) identify one or more matching reads at respective locations within the pair of adjacent tiles, and (ii) determine a pairwise offset for the pair of adjacent tiles based on the respective locations of the one or more matching reads, determine a swath offset for the pair of overlapping swaths based on the pairwise offsets for the respective pairs of adjacent tiles in the pair of overlapping swaths, and stitch the respective pairs of adjacent tiles together by aligning the coordinate systems of the respective tiles using the swath offset to generate a continuous coordinate system across the pair of overlapping swaths.

Example 9. The system of example 8, wherein the pair of overlapping swaths includes a first swath and a second swath, and wherein to stitch respective pairs of adjacent tiles together, the computer-readable instructions cause the system to: apply a transformation to the coordinate systems of tiles in the second swath based on the swath offset; determine a middle portion of the continuous coordinate system between the first and second swaths; and filter one or more duplicated reads from the continuous coordinate system, wherein a duplicated read is at a location within the continuous coordinate system on an opposite side of the middle portion from the first or second swath where the duplicated read was identified before the transformation.

Example 10. The system of example 8 or example 9, wherein the respective tiles within the pair swaths are overlapping, wherein the pair of overlapping swaths are adjacent horizontally such that respective pairs of adjacent tiles in the pair of overlapping swaths are adjacent horizontally, the pairwise offset is a horizontal pairwise offset, the swath offset is a horizontal swath offset, and the computer-readable instructions further cause the system to: for respective pairs of vertically adjacent tiles within a particular swath: (i) identify one or more matching reads at respective locations within the pair of vertically adjacent tiles, and (ii) determine a vertical pairwise offset for the pair of vertically adjacent tiles based on the respective locations of the one or more matching reads, determine a vertical swath offset for the particular swath based on the vertical pairwise offsets for the respective pairs of vertically adjacent tiles in the particular swath, and stitch the respective pairs of vertically adjacent tiles together by aligning the coordinate systems of the respective tiles using the vertical swath offset.

Example 11. The system of any of examples 8 to 10, wherein the computer-readable instructions further cause the system to: rotate a coordinate system for at least one of the plurality of tiles within the particular swath to align the coordinate system of the tile with coordinate systems for other tiles within the particular swath.

Example 12. The system of any of examples 8 to 11, wherein respective tiles are generated for a particular cycle of a plurality of cycles, and wherein to rotate the coordinate system for a respective tile, the instructions cause the system to: select a desired cycle for use in aligning the coordinate systems of the respective tiles, and align the coordinate systems of the respective tiles to a coordinate system for the desired cycle by applying an affine transformation for the desired cycle.

Example 13. The system of any of examples 8 to 12, wherein to align the coordinate systems of the respective tiles, the instructions cause the system to: for the respective tiles: determine, for the desired cycle, one or more of: (i) a translation parameter, (ii) a magnification parameter, or (iii) a shear mapping parameter; determine a center location of the respective tile; identify locations of clusters of reads within the respective tile; and for respective cluster locations, apply, to a difference between the respective cluster location and the center location, one or more of: (i) the translation parameter, (ii) the magnification parameter, or (iii) the shear mapping parameter.

Example 14. The system of any of examples 8 to 13, wherein the instructions further cause the system to at least one of: identify a genomic sequence at a particular location using the continuous coordinate system; identify a genetic variant based on the genomic sequence; or identify a phenotype associated with the genetic variant.

Example 15. A tangible, non-transitory computer-readable medium storing instructions for stitching genomic sequence data that, when executed by one or more processors, cause the one or more processors to: obtain tile data for respective tiles in the pair of overlapping swaths of genomic sequence data, wherein the tile data is a dataset corresponding to a region of a sample, the dataset including a coordinate system and a set of reads of genomic sequences at locations within the coordinate system, for respective pairs of adjacent tiles in the pair of overlapping swaths: (i) identify one or more matching reads at respective locations within the pair of adjacent tiles, and (ii) determine a pairwise offset for the pair of adjacent tiles based on the respective locations of the one or more matching reads, determine a swath offset for the pair of overlapping swaths based on the pairwise offsets for the respective pairs of adjacent tiles in the pair of overlapping swaths, and stitch the respective pairs of adjacent tiles together by aligning the coordinate systems of the respective tiles using the swath offset to generate a continuous coordinate system across the pair of overlapping swaths.

Example 16. The computer-readable medium of example 15, wherein the pair of overlapping swaths includes a first swath and a second swath, and wherein to stitch respective pairs of adjacent tiles together, the instructions cause the one or more processors to: apply a transformation to the coordinate systems of tiles in the second swath based on the swath offset; determine a middle portion of the continuous coordinate system between the first and second swaths; and filter one or more duplicated reads from the continuous coordinate system, wherein a duplicated read is at a location within the continuous coordinate system on an opposite side of the middle portion from the first or second swath where the duplicated read was identified before the transformation.

Example 17. The computer-readable medium of example 15 or example 16, wherein the respective tiles within the pair of swaths are overlapping, wherein the pair of overlapping swaths are adjacent horizontally such that respective pairs of adjacent tiles in the pair of overlapping swaths are adjacent horizontally, the pairwise offset is a horizontal pairwise offset, the swath offset is a horizontal swath offset, and the instructions further cause the one or more processors to: for respective pairs of vertically adjacent tiles within a particular swath: (i) identify one or more matching reads at respective locations within the pair of vertically adjacent tiles, and (ii) determine a vertical pairwise offset for the pair of vertically adjacent tiles based on the respective locations of the one or more matching reads, determine a vertical swath offset for the particular swath based on the vertical pairwise offsets for the respective pairs of vertically adjacent tiles in the particular swath, and stitch the respective pairs of vertically adjacent tiles together by aligning the coordinate systems of the respective tiles using the vertical swath offset.

Example 18. The computer-readable medium of any of examples 15 to 17, wherein the instructions further cause the one or more processors to: rotate a coordinate system for at least one of the plurality of tiles within the particular swath to align the coordinate system of the tile with coordinate systems for other tiles within the particular swath.

Example 19. The computer-readable medium of any of examples 15 to 18, wherein respective tiles are generated for a particular cycle of a plurality of cycles, and wherein to rotate the coordinate system for the tile, the instructions cause the one or more processors to: select a desired cycle for use in aligning the coordinate systems of the respective tiles, and align the coordinate systems of the respective tiles to a coordinate system for the desired cycle by applying an affine transformation for the desired cycle.

Example 20. The computer-readable medium of any of examples 15 to 19, wherein to align the coordinate systems of respective tiles, the instructions cause the one or more processors to: for the respective tiles: determine, for the desired cycle, one or more of: (i) a translation parameter, (ii) a magnification parameter, or (iii) a shear mapping parameter; determine a center location of the respective tile; identify locations of clusters of reads within the respective tile; and for respective cluster locations, apply, to a difference between the respective cluster location and the center location, one or more of: (i) the translation parameter, (ii) the magnification parameter, or (iii) the shear mapping parameter.

### Exemplary Sequencing Platform Environment

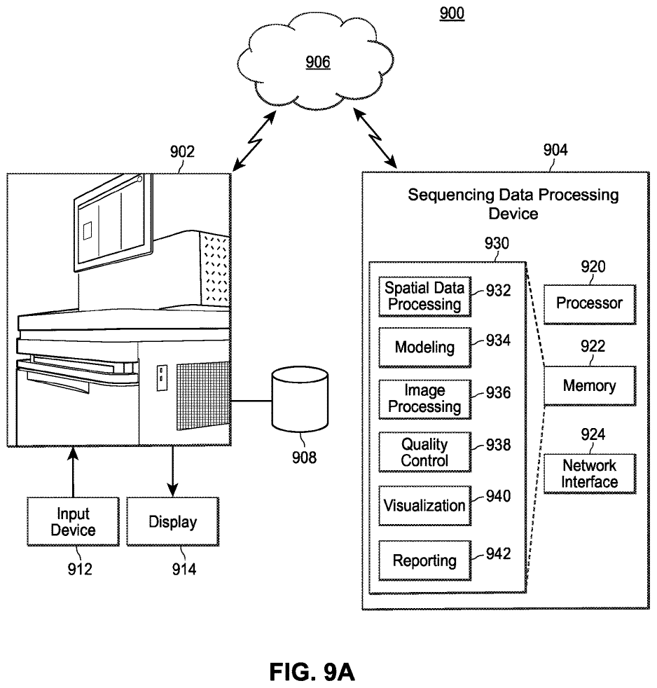

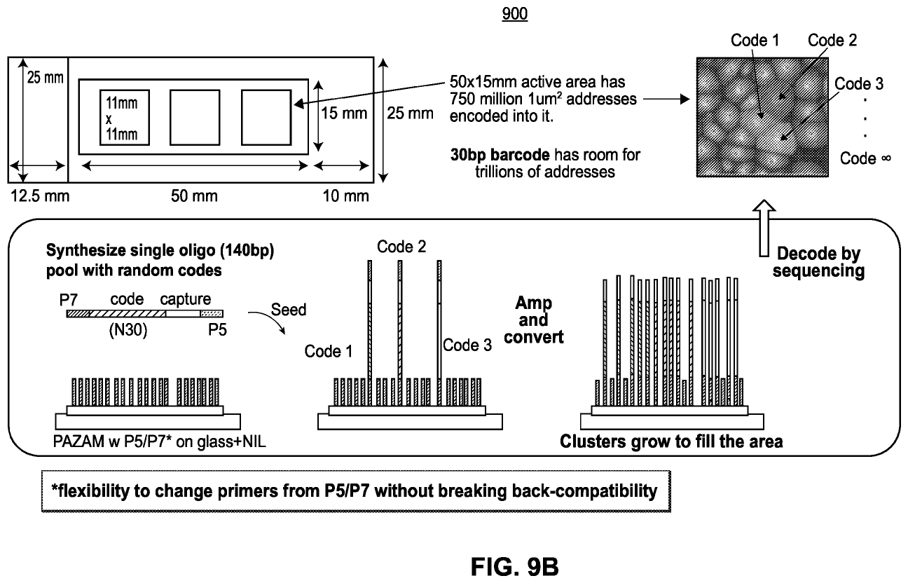

FIG. 9A depicts an exemplary sequencing platform environment 900 for performing the present techniques, according to some aspects. The environment 900 may include computing resources for improving quality control of spatial transcriptomics substrates. The exemplary sequencing platform environment 900 may include a sequencing platform 902, a sequencing data processing device 904, an electronic network 906 and an electronic database 908.

The sequencing platform 902 may be an Illumina NovaSeq 6000, in some aspects. The sequencing platform 902 may include an input device 912 and a display device 914 that enable a user to interact with the sequencing platform 902. Illumina is a leading company in the field of genetic sequencing technologies. They are known for developing and manufacturing high-throughput DNA sequencing platforms and associated reagents, software, and consumables. Illumina's machines have played a significant role in advancing genomics research and have been widely adopted in various scientific and clinical applications. Illumina sequencing machines use a technique called massively parallel sequencing or next-generation sequencing (NGS). This approach enables the simultaneous sequencing of millions of DNA fragments, providing researchers with vast amounts of genomic data at a relatively low cost and high speed compared to traditional sequencing methods.

NovaSeq is one of Illumina's notable sequencing platforms, and represents a significant advancement in sequencing technology. It offers high throughput and scalability, enabling various applications ranging from small targeted panels to large-scale whole-genome sequencing. NovaSeq systems are known for their efficiency and cost-effectiveness. Illumina machines generally utilize a sequencing-by-synthesis approach. Fragments of DNA are attached to a solid surface and amplified into clusters. Fluorescently labeled nucleotides are then sequentially added, and the emitted light signals are captured and processed to determine the DNA sequence. Thus, the sequencing platform 902 may generate fluorescent imagery (e.g., heatmaps) corresponding captured light signals.

In some aspects, the sequencing platform 902 may further perform spatial barcoding of a substrate, as depicted in FIG. 9B. In the present techniques, the substrate may include flow cells, genotyping arrays, a microarray, a substrate with capture oligos, a substrate with molecules, any array that has capture molecules, any array used for sequencing, etc. Specifically, spatial transcriptomics is a technique that combines traditional gene expression analysis with spatial information. In particular, the sequencing platform 902 may map gene expression patterns within the context of tissue or cellular organization of a substrate. Instead of measuring gene expression levels in bulk tissue, spatial transcriptomics allows researchers to determine where specific genes are expressed within the tissue sample by preserving the spatial information of the tissue while simultaneously capturing the gene expression profile.

In some aspects, the sequencing platform 902 achieves preservation of spatial information by integrating spatially barcoded oligonucleotides or probes into the tissue sample during preparation, as shown in FIG. 9B. These barcodes or probes carry unique identifiers that can be linked to specific genes or transcripts. The tissue may then be processed and sequenced using high-throughput sequencing technologies (e.g., by the sequencing platform 902) that provide corresponding data to the sequencing data processing device 904 for further processing/analysis, as described below. For example, during sequencing, spatially-barcoded probes may capture the gene expression information and their corresponding spatial location in the tissue. The sequencing data processing device 904 may analyze the resulting data to generate spatial maps that illustrate the distribution and activity of genes within the tissue sample. This information advantageously helps researchers understand the cellular heterogeneity, tissue organization, and interactions between different cell types within the tissue. For example, spatial transcriptomics has various applications in biological and biomedical research, such as identifying cell types and subtypes within complex tissues, unraveling spatial gene expression patterns in development and disease, and uncovering the interactions between cells in a tissue microenvironment.

In general, the sequencing platform 902 may provide high-throughput sequencing using a combination of flow cells, cluster generation instructions, imaging/sequencing and data analysis. For example, the sequencing platform 902 may include flow cells in which sequencing reactions take place. Each of the flow cells may include a respective array of microscopic wells (“nanowells”) that capture the DNA fragments for sequencing. Each nanowell may include one or more oligonucleotide primers for DNA synthesis during sequencing. The sequencing platform 902 may perform cluster generation, a process involving amplification of DNA fragments into clusters on the flow cell. The flow cell may be patterned, wherein a plurality of clusters of DNA molecules are spatially organized into defined positions, preserving information during the sequence-by-synthesis process. The sequencing platform 902 may include imaging and sequencing operations, such as a scanner for capturing high-resolution images of one or more clusters on a flow cell, and for adding fluorescent labels to nucleotides on flow cells. The sequencing platform 902 may include instructions for capturing signals emitted by fluorescent labels and processing them into raw sequence data (i.e., sequence reads). The raw sequence data may be further processed to generate data processed by the spatial data processing module 932, for example.

In some aspects, the sequencing data processing device 904 may be implemented as one or more computing devices (e.g., one or more servers, one or more laptops, one or more mobile computing devices, one or more tablets, one or more wearable devices, one or more cloud-computing virtual instances, etc.). The sequencing data processing device 904 may include one or more processors 920, one or more memories 922 and a network interface controller 924 including one or more Ethernet network interface controllers, one or more wireless network interface controllers, etc., and in some aspects, having advanced features such as hardware acceleration, specialized networking protocols, etc.

In some aspects, the one or more processors 920 may include one or more central processing units, one or more graphics processing units, one or more field-programmable gate arrays, one or more application-specific integrated circuits, one or more tensor processing units, one or more digital signal processors, one or more neural processing units, one or more RISC-V processors, one or more coprocessors, one or more specialized processors/accelerators for artificial intelligence or machine learning-specific applications, one or more microcontrollers, etc.

The one or more memories 922 may include volatile and/or non-volatile storage media. For example, the one or more memories 922 may include one or more random access memories, one or more read-only memories, one or more cache memories, one or more hard disk drives, computer-readable medium, one or more solid-state drives, one or more non-volatile memory express, one or more optical drives, one or more universal serial bus flash drives, one or more external hard drives, one or more network-attached storage devices, one or more cloud storage instances, one or more tape drives, non-transitory computer-readable media, etc. The one or more memories 922 may have stored thereon one or more modules 930, for example, as one or more sets of computer-executable instructions.

In some aspects, the modules 930 may include additional storage, such as one or more operating systems (e.g., Microsoft Windows, GNU/Linux, Mac OSX, etc.). The operating systems may be configured to run the modules 930 during operation of the sequencing platform 902—for example, the modules 930 may include additional modules and/or services for receiving and processing data from the sequencing platform 902. The modules 930 may be implemented using any suitable computer programming language(s) (e.g., Python, JavaScript, C, C++, Rust, C#, Swift, Java, Go, LISP, Ruby, Fortran, etc.). The memories may be non-transitory memories.

The modules 930 may include a spatial data processing module 932. The spatial data processing module 932 may process data from the sequencing platform 902 to enable further processing of spatial transcriptomics as discussed above. For example, the modules 930 may include a library of software (e.g., a set of computer-executable instructions that, when executed, cause the sequencing data processing device 904 to perform actions). For example, the software/instructions may include receiving data that correspond to outputs from the sequencing platform 902. The spatial data processing module 932 may include instructions for preprocessing, normalizing and/or storing the received data (e.g., in the electronic database 908 and/or in the one or more memories 922). In some aspects the spatial data processing module 932 may be used to determine metrics to quantify variability in spatial distributions (e.g., VMR, as discussed herein).

The modules 930 may include a modeling module 934. The modeling module 934 may include instructions for converting measurements from cycles, tiles, bins, flow cells and other data into data structure objects. For example, the modeling module 934 may include a set of computer-executable instructions that, when executed, accept a raw data stream (e.g., from the sequencing platform 902) and transform the raw data into structured data, such as a tile. The modeling module 934 may include instructions for generating nested data structures (e.g., an hierarchical model) or composed data structures (e.g., one or more models that are included, or nested, within other models). In some aspects, the modules 930 may include a data mapper that transforms raw data into data structures (e.g., objects) and then further transforms objects into database constructs. For example, the data mapper may be an object-relational mapper that receives raw data and generates objects, and stores those objects in the electronic database 908. In still further aspects, the data mapper may store the objects as serialized code objects, such as JSON objects in the one or more memories 922. The transformed data structures may be linear data structures, recursive data structures, etc.

The modules 930 may further include an image processing module 936 that reads and/or generates digital image data. For example, in some aspects, the image processing module 936 may receive image data from the sequencing platform 902. In some aspects, as noted, the sequencing platform 902 may transmit (e.g., via the network 906) or otherwise provide image data to the sequencing data processing device 904. The image processing module 936 may process the image data, for example, to generate heatmaps, histograms or other visualizations from raw image data. Herein “images” may refer to digital image data, such as binary data in an open source or proprietary data format, including video data, still image data, stereoscopic image data, etc. In some aspects, the image processing module 936 may generate a plurality of bins corresponding to an image tile, by subdividing the tile into the plurality of bins. The bins may be represented in the one or more memories 922 as being associated with the subdivided image tile. This one-to-many relationship may be stored in the electronic database 908, in some aspects.

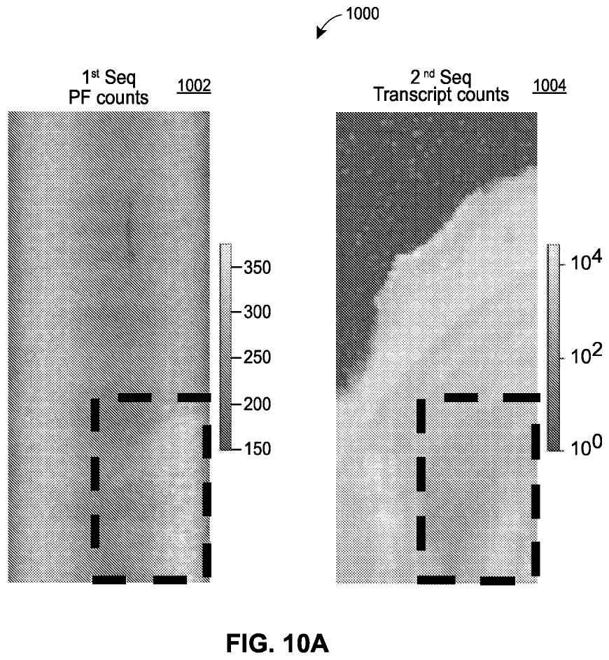

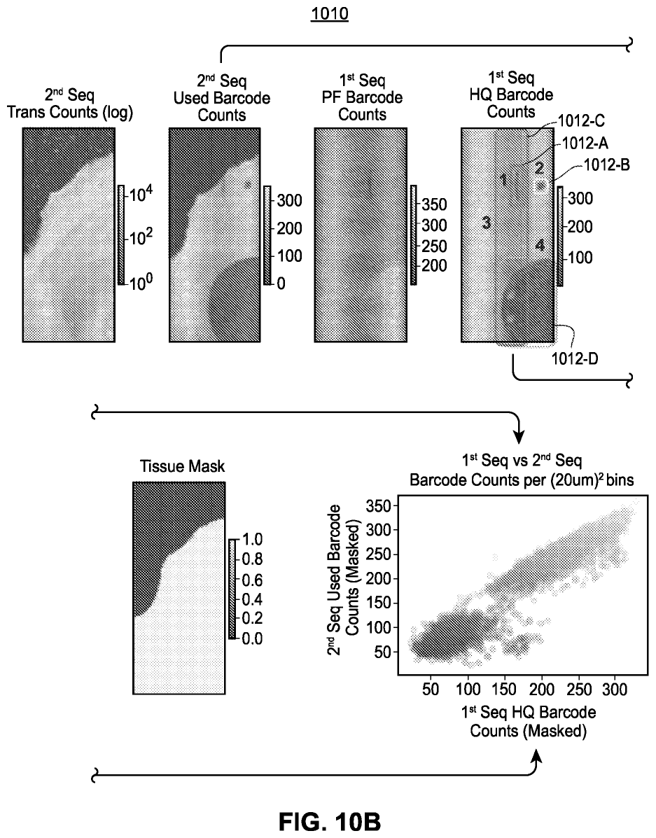

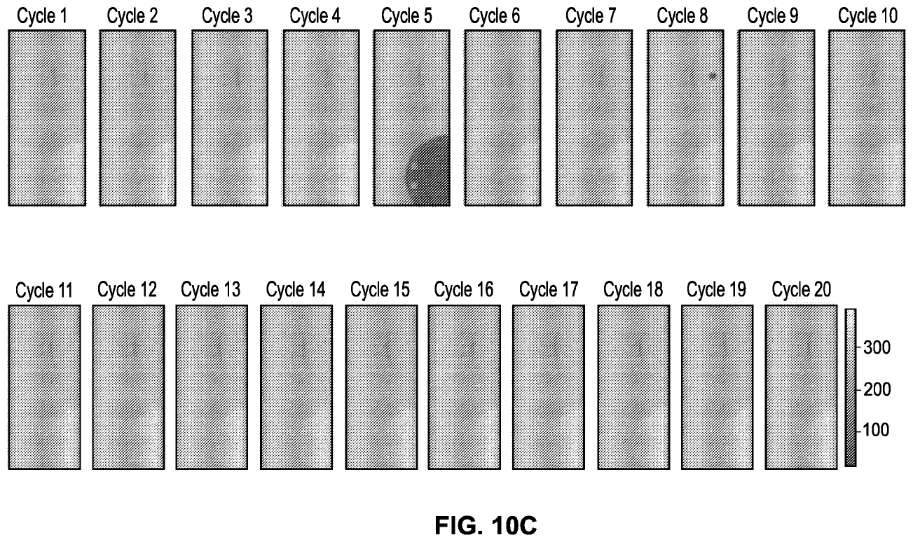

The modules 930 may include a quality control module 938. The quality control module 938 may include computer-executable code for performing functions such as processing images (e.g., images received by the image processing module 936). For example, the quality control module 938 may process images/image data received by the sequencing data processing device 904 from the 902. The quality control module 938 may process the image data including any relationships to subdivisions or other relationships. The quality control module 938 may include a set of computer-executable instructions that, when executed, cause the sequencing data processing device 904 to compute one or more quality control scores. In some aspects, the quality control module 938 may determine one or more regions in the image data that include artifacts, such as those depicted in FIG. 10B (depicted as encircled areas 1-4).

In some aspects, the quality control module 938 may perform data preprocessing, wherein, for example, raw sequencing data is processed in various ways. For example, the 938 may trim adapter sequences, filter out low-quality reads and/or align reads to a reference genome or transcriptome. Additional preprocessing operations may be performed, such as differential expression analysis, functional analysis, variant calling, quantification, etc.

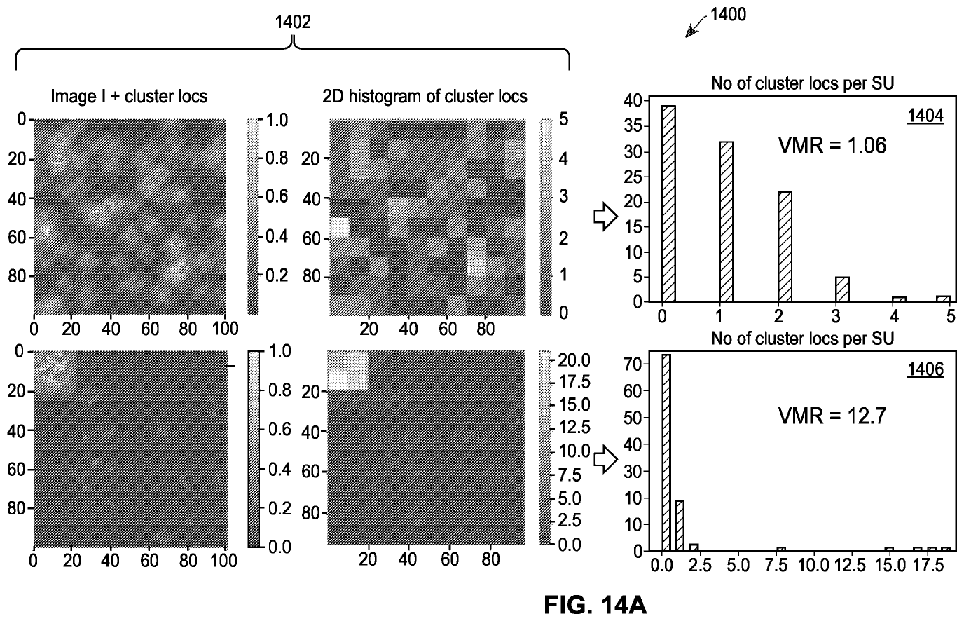

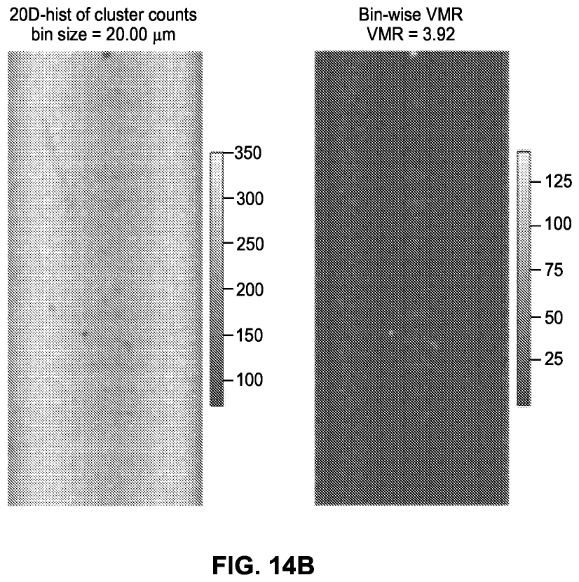

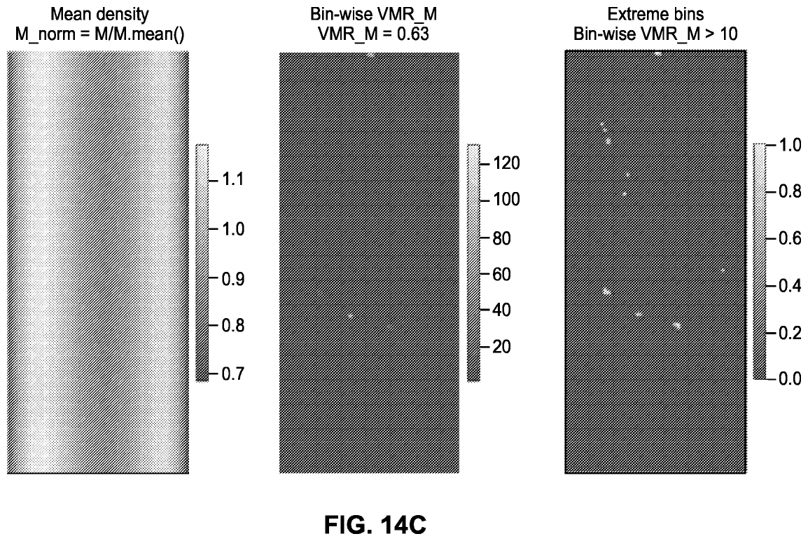

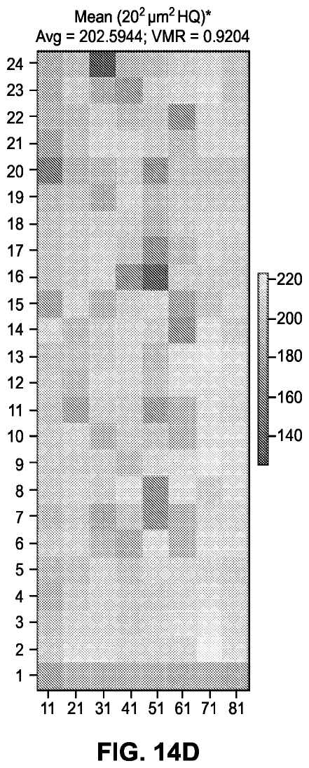

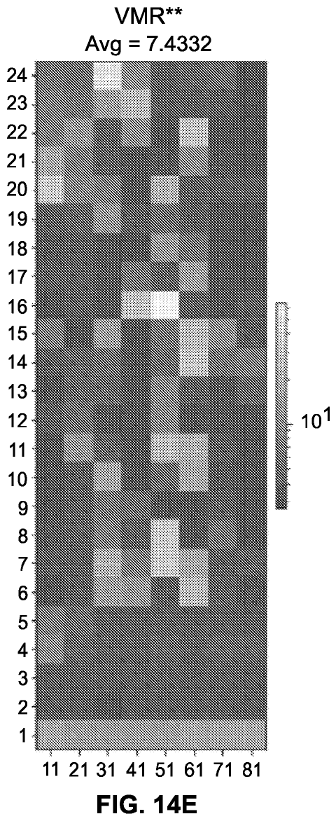

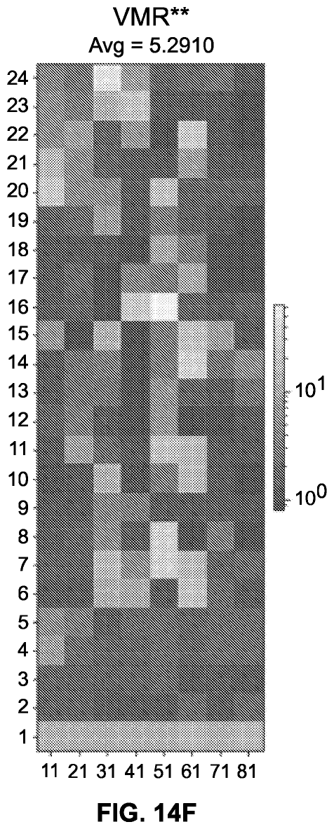

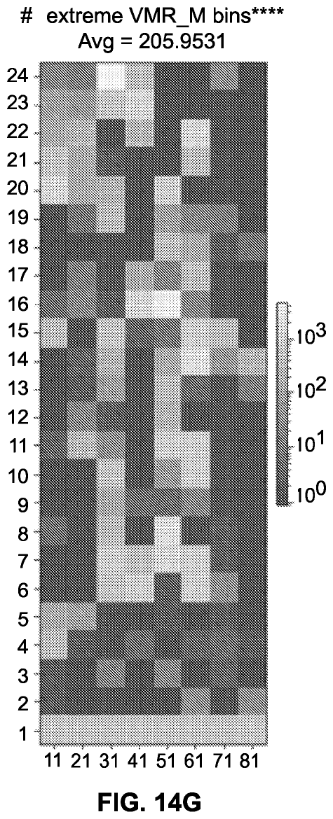

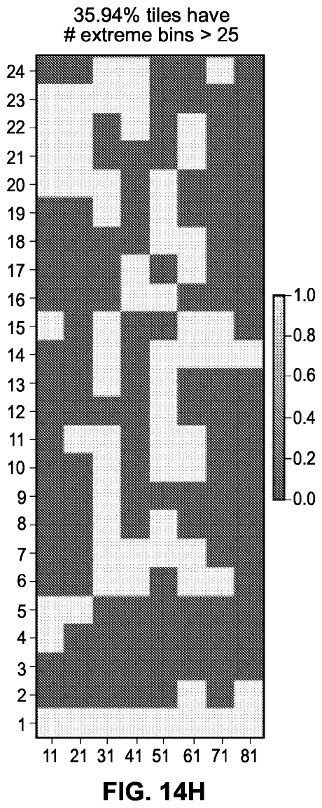

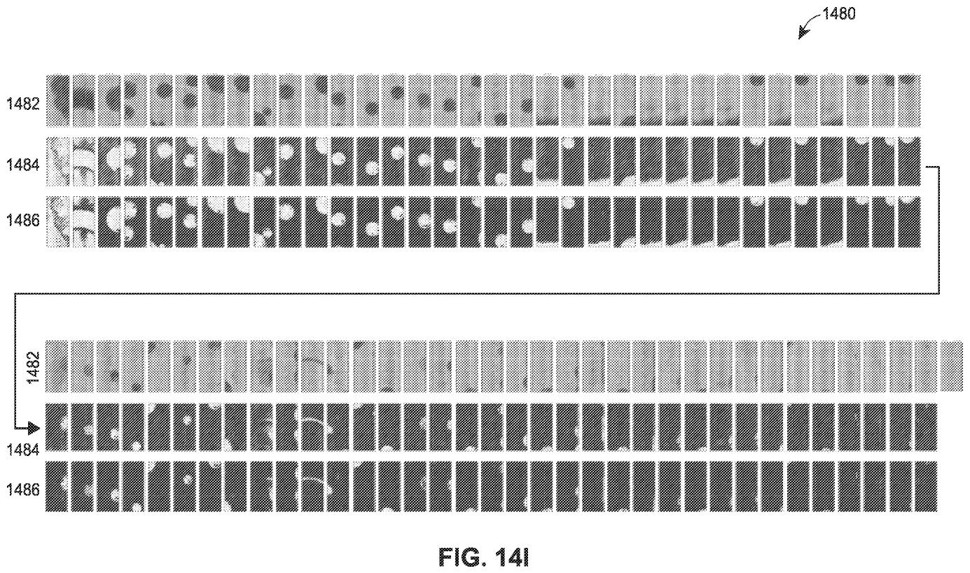

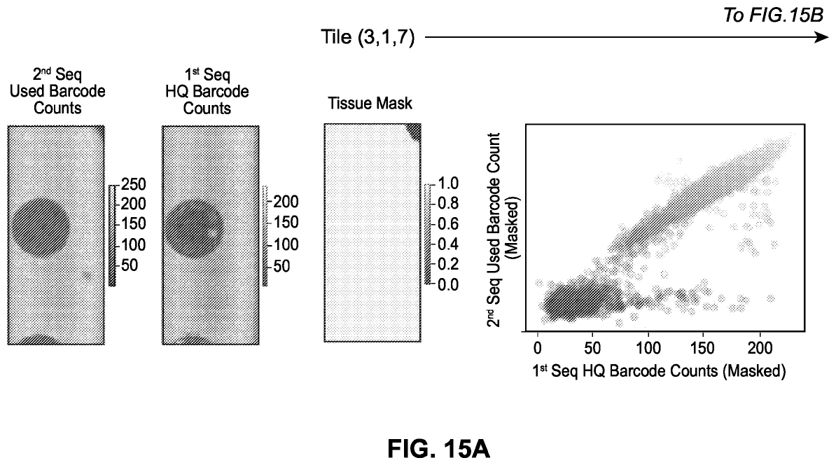

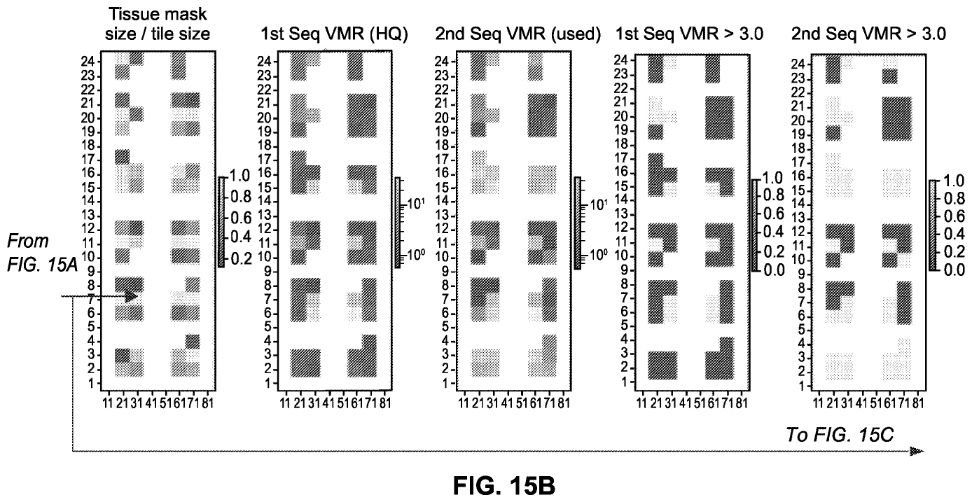

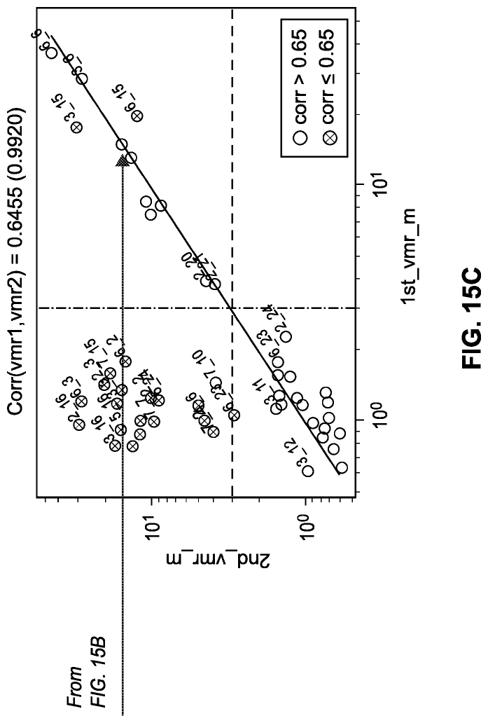

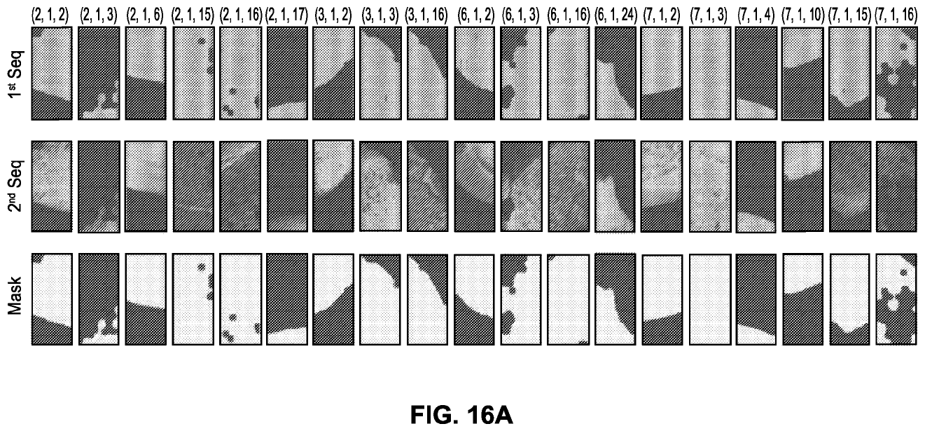

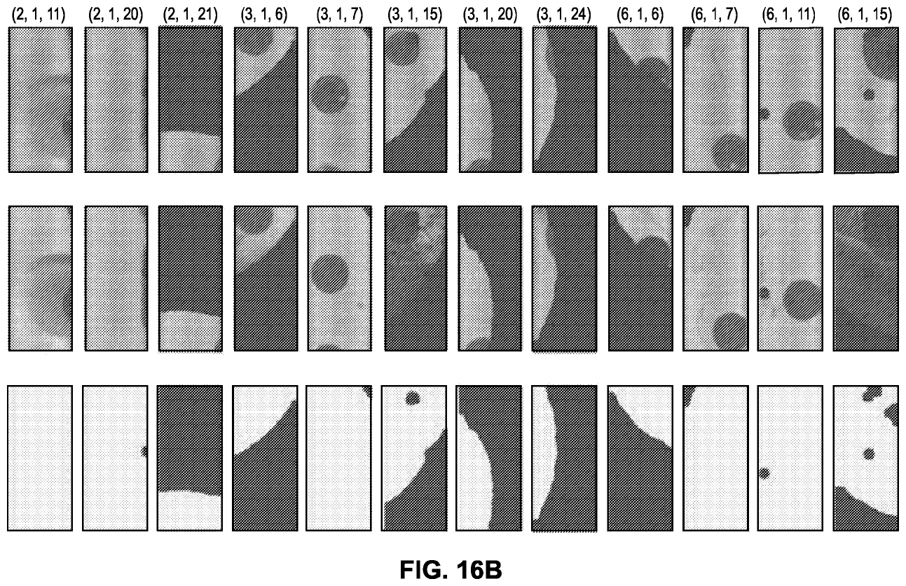

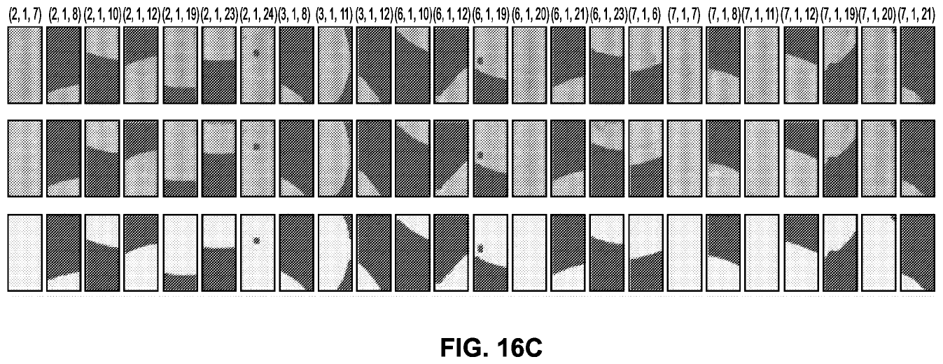

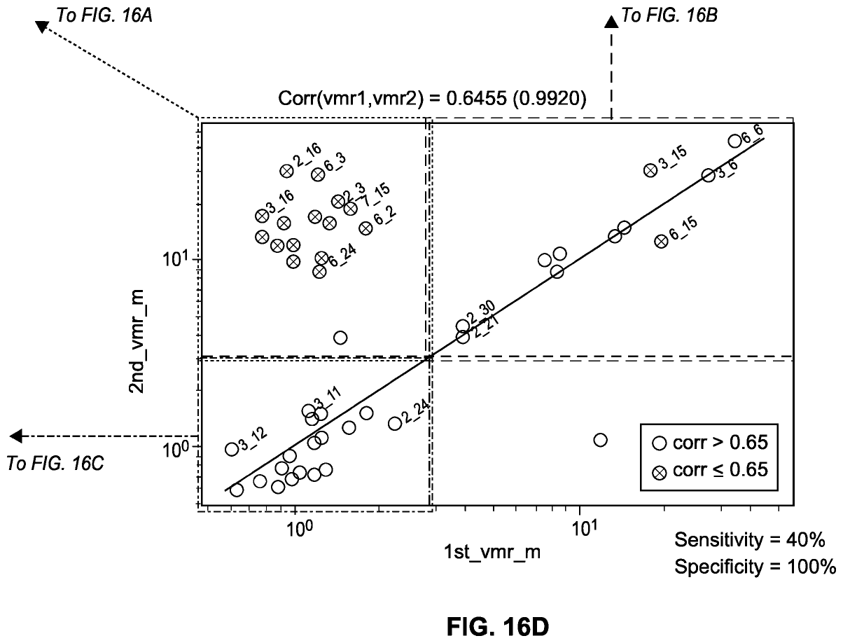

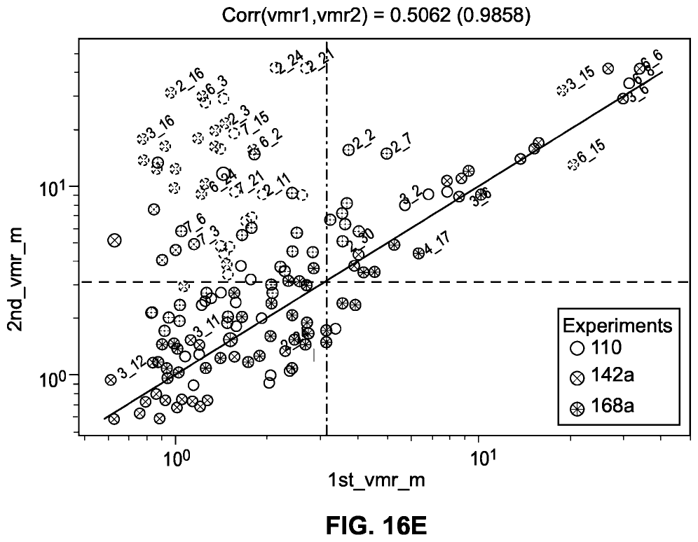

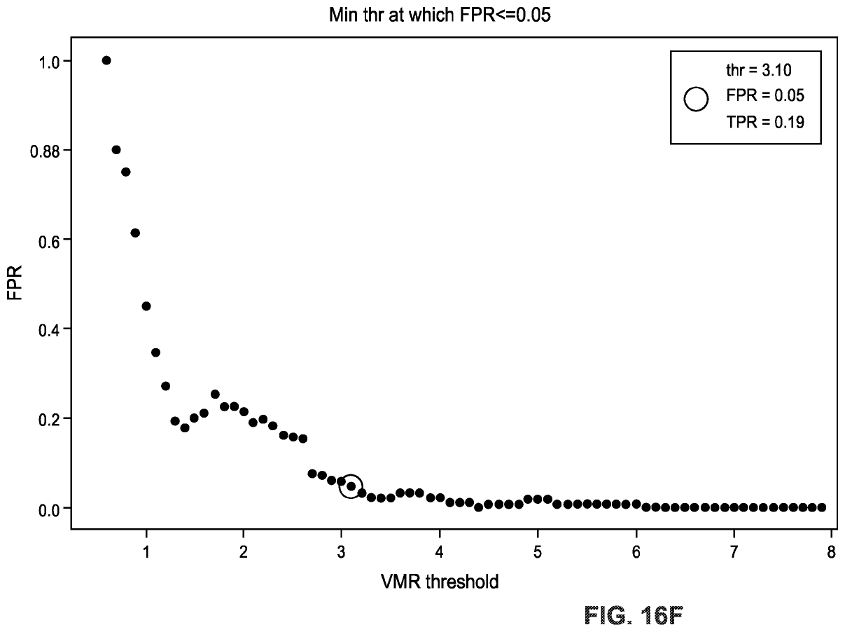

The modules 930 may include a visualization module 940. The visualization module 940 may include instructions for generating one or more sets of computer-executable instructions for generating visualizations of sequencing data (including raw data and processed data). For example, the visualization module 940 may include a library of instructions for generating a heatmap as shown in FIG. 14A, below. In some aspects, the visualization module 940 may include a library of instructions for generating a histogram (e.g., a two-dimensional (2D) histogram) of cluster locations. The visualization module 940 may include a library of instructions for generating a plot such as the plot of used barcode counts vs. high-quality barcode counts in FIG. 15A, below. The visualization module 940 may include instructions for incorporating a tissue mask into visualizations, in some aspects. In some aspects, the visualization module 940 may include instructions for generating correlation charts, such as the chart depicted in FIG. 16E, below. In some aspects, the visualization module 940 may include instructions for generating scatter plots, such as the scatter plot chart depicted in FIG. 16F, below. In some aspects, the visualization module 140 may include generating artifact identifications, such as those depicted in FIG. 10B (depicted as encircled areas 1-4).

The modules 930 may include a reporting module 942. The reporting module 942 may include computer-executable instructions for generating one or more reports corresponding to the sequencing platform 902 and the sequencing data processing device 904. For example, the reporting module 942 may generate a digital report (e.g., an electronic document) that includes a description of the sequencing data received by the sequencing data processing device 904 and quality control performed by the sequencing data processing device 904. For example, the digital report may indicate whether the data corresponding to the substrate of the sequencing platform 902 and processed by the sequencing data processing device 904 indicates that the substrate is usable by a downstream process (i.e., is a QC pass) or should be discarded or mitigated (i.e., is a QC fail). The reporting module 942 may include outputs generated by the visualization module 940, such as graphical depictions of substrates processed by the sequencing platform 902.

In some aspects, some or all of the sequencing data processing device 904 may be integrated into the sequencing platform 902. For example, the entirety of the sequencing data processing device 904 may be integrated as hardware and/or software that is part of the sequencing platform 902. In some aspects, only one or more of the modules 930 may be so integrated. The modules 930 may be configured to communicate with one another (e.g., via inter-process communication, via a bus, message queue, sockets, etc.).

The electronic network 906 may communicatively couple the elements of the environment 900. The network 906 may include public network(s) such as the Internet, a private network such as a research institution or corporation private network, and/or any combination thereof. The network 906 may include a local area network (LAN), a wide area network (WAN), a cellular network, a satellite network, and/or other network infrastructure, whether wireless or wired. In some aspects, the network 906 may be communicatively coupled to and/or part of a cloud-based platform (e.g., a cloud computing infrastructure). The network 906 may utilize communications protocols, including packet-based and/or datagram-based protocols such as Internet protocol, transmission control protocol, user datagram protocol, and/or other types of protocols. The network 906 may include one or more devices that facilitate network communications and/or form a hardware basis for the networks, such as one or more switches, one or more routers, one or more gateways, one or more access points (such as a wireless access point), one or more firewalls, one or more base stations, one or more repeaters, one or more backbone devices, etc.

The electronic database 908 may include one or more suitable electronic databases for storing and retrieving data, such as relational database (e.g., MySQL databases, Oracle databases, Microsoft SQL Server databases, PostgreSQL databases, etc.). The electronic database 908 may be a NoSQL database, such as a key-value store, a graph database, a document store, etc. The electronic database 908 may be an object-oriented database, a hierarchical database, a spatial database, a time-series database, an in-memory database, etc. In some aspects, some or all of the electronic database 908 may be a distributed database.

In operation, the exemplary sequencing platform environment 900 may allow one or more samples (e.g., DNA sample, RNA samples, tissue sample, etc.) to be loaded into the sequencing platform 902 (e.g., via the sequencing platform 902). Preparing these samples for analysis may include DNA extraction, library preparation and quality control to ensure the samples are suitable for sequencing. The prepared DNA or RNA samples may be converted into sequencing libraries, which may include fragmenting the genetic material, attaching adapters, amplifying the sequences, and tagging the sequences with unique identifier(s). Next, cluster generation may be performed to generate pluralities of clusters of the samples on a flow cell. Following cluster generation, the exemplary sequencing platform environment 900 may perform sequencing-by-synthesis to sequence each cluster. This may include emitting fluorescence that may be captured as digital images by the sequencing platform 902, and the generation of raw sequencing data (i.e., reads). The raw sequencing data may be stored in the electronic database 908 and/or transmitted (e.g., via the network 906) to the sequencing data processing device 904. The sequencing data processing device 904 may process the raw sequencing data reads using the techniques discussed herein, to process the raw sequencing data into structured data and/or determine quality control of processed data. The sequenced data may be visualized, stored, transmitted (i.e., via email), and/or reported (e.g., via generation of one or more reports).

### Exemplary Computer-Implemented Visualization Experiment Aspects

FIG. 10A depicts heatmaps 1000 showing pass filter counts 1002 and transcript counts 1004, according to some aspects. Herein, heatmaps may be generated by the visualization module 940. Generally, a “heatmap” is a graphical representation of gene expression patterns. Herein, generally, gene expression patterns may be depicted in heatmaps corresponding to second seq processes, while spatial patterns of barcodes seeded on a substrate may be depicted in heatmaps corresponding to first seq processes. Heatmaps in the present techniques generally depict 2D spatial histograms, wherein each bin represents counts of barcodes (1st seq) or expressed genes (2nd seq). In some aspects, rows/columns correspond to different spatial x,y coordinates in a 2D spatial depiction of a substrate. Colors in the heatmap may indicate the expression levels, with brighter colors indicating higher expression and darker colors indicating lower expression.

The pass filter counts 1002 may represent a number of sequencing reads that have passed quality control filters and are considered valid for further analysis. As noted, sequencing machines produce a large number of raw reads, but not all of them are of sufficient quality for downstream analysis. Pass filter counts reflect the reads that meet quality criteria and are thus reliable for subsequent steps in genomics analysis. The transcript counts 1004 may represent a quantification of the number of mRNA transcripts of specific genes present in a sample. Generally, transcript counts are used to measure gene expression levels, providing insights into which genes are active or expressed in a particular biological sample.

Further, as noted, the quality of spatial molecular maps produced after downstream processes (e.g., second seq) relies on a uniform distribution of spatial barcodes across upstream processes (e.g., first seq flow cells). Regions of variable cluster density and quality in the upstream process can lead to nonuniform spatial sensitivity, thus producing damaged spatial molecular maps with various local artifacts (e.g., scratches, bubbles, etc.) undermining ultimate product quality. The present techniques advantageously enable nonuniformity in spatial sensitivity to be quantified, which directly enables prediction of artifacts in downstream processes based on upstream processes; determination of usability of particular upstream flow cells; accounting for artifacts in downstream analysis by suitably normalizing downstream processes; and quality improvement of downstream processes.

FIG. 10B depicts heatmaps 1010 showing example artifacts 1012 that can be predicted, and correlations between spatial density of barcodes used and spatial density of high-quality barcodes, according to some aspects. For example, the present techniques enable detection of variable spatial sensitivity in downstream processes (e.g., second seq) by capturing spatial density of barcodes used in downstream processes better than by the spatial density of second sequence unique molecular identifier (UMI) counts (i.e., transcript counts).

Regions lacking high-quality barcodes in the first experiment contain artifacts, and these artifacts continue to be observable in the data from the second experiment. Further, artifacts in downstream experiments can be predicted from upstream sequencing data. For example, high quality barcode data has a Phred Q score of average error probability over C cycles >x, i.e.,

$$- 10 {log}_{10} \frac{1}{c} {\sum }_{c} {10}^{- {Q}_{c} / 10} > x .$$

Where each high quality cluster in cycle c has corresponding Q_{c} score>x. For example, x may be a value (e.g., 30) in some aspects.

FIG. 10C depicts sequencing cycle heatmaps, according to some aspects. In the context of genomics sequencing, “cycles” typically refer to sequencing cycles or sequencing rounds. These cycles are the repeated steps of adding and identifying nucleotides in the sequencing process. Each cycle generally includes respective steps of denaturation, annealing, extension, and imaging (e.g., using fluorescence). The present techniques may perform many (hundreds, thousands or more) of cycles to read protein sequences and generate data for processing (e.g., by sequencing data processing device 904). In this context, each cycle may lead to one nucleotide of the barcodes sequenced in the first sequencing step.

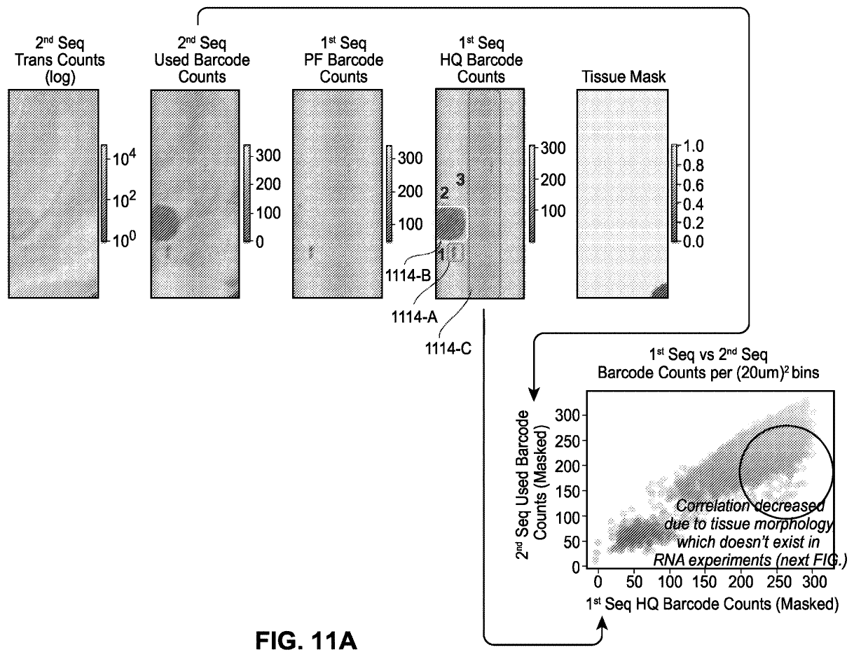

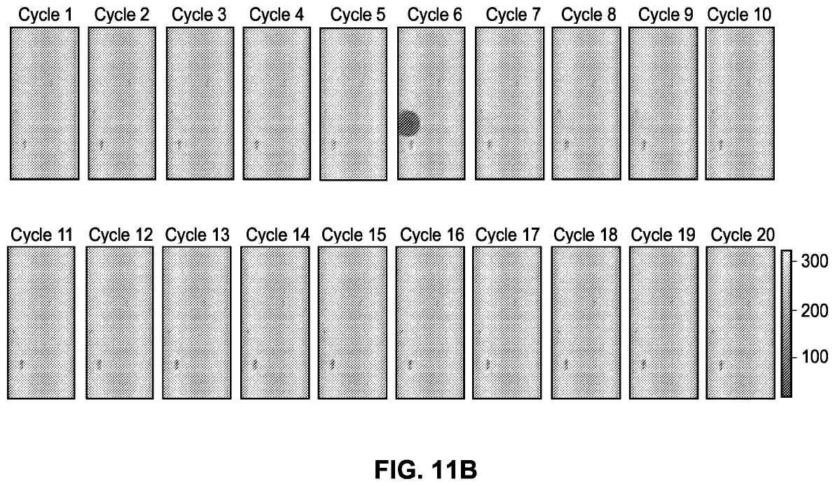

FIG. 11A depicts heatmaps showing example artifacts 1114A-1114C that can be predicted, and correlations between spatial density of barcodes used and spatial density of high-quality barcodes, according to some aspects. In particular, FIG. 11A depicts decreased correlations due to tissue morphology. In FIG. 11A, high quality cluster counts in each barcode cycle of an upstream process (e.g., first seq) may have a high quality cluster Q score of >30, while high quality barcodes may have a Q score of average error probability >30. FIG. 11B depicts sequencing cycle heatmaps corresponding to FIG. 11A, according to some aspects.

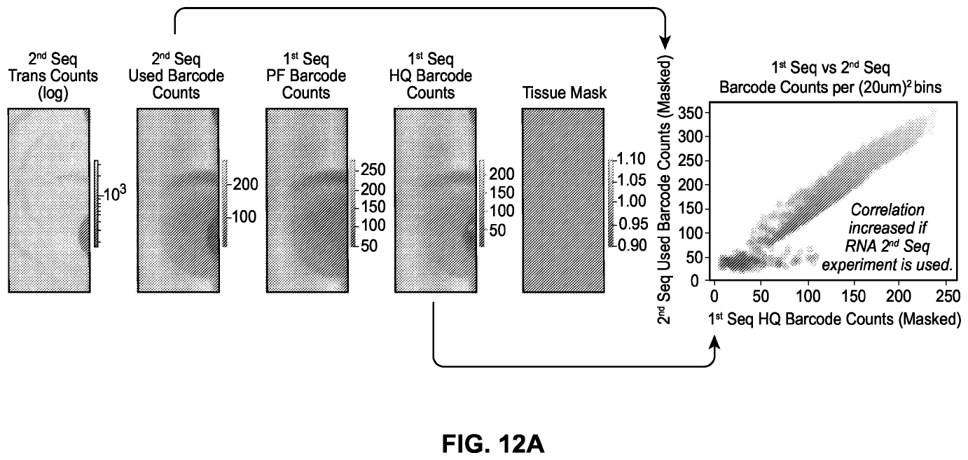

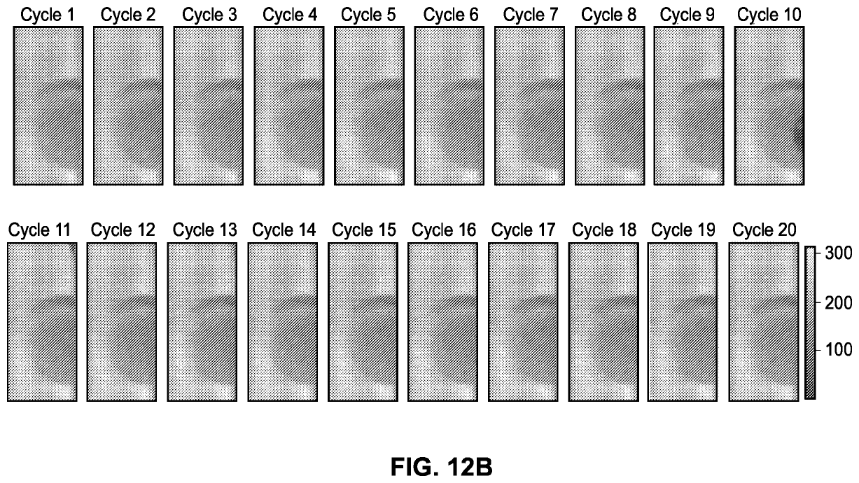

FIG. 12A depicts heatmaps showing example artifacts that can be predicted, and correlations between spatial density of barcodes used and spatial density of high-quality barcodes for an RNA experiment, according to some aspects. In FIG. 12A, high quality cluster counts in each barcode cycle of an upstream process (e.g., first seq) may have a high quality cluster Q score (e.g., >30), while a high quality barcode has a Q score average error probability (e.g., of >30). FIG. 12B depicts sequencing cycle heatmaps corresponding to FIG. 12A, according to some aspects.

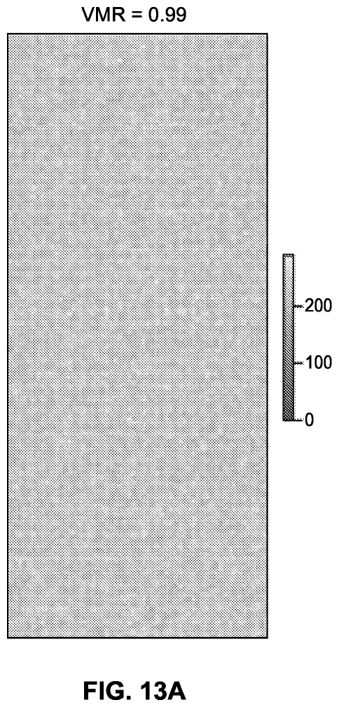

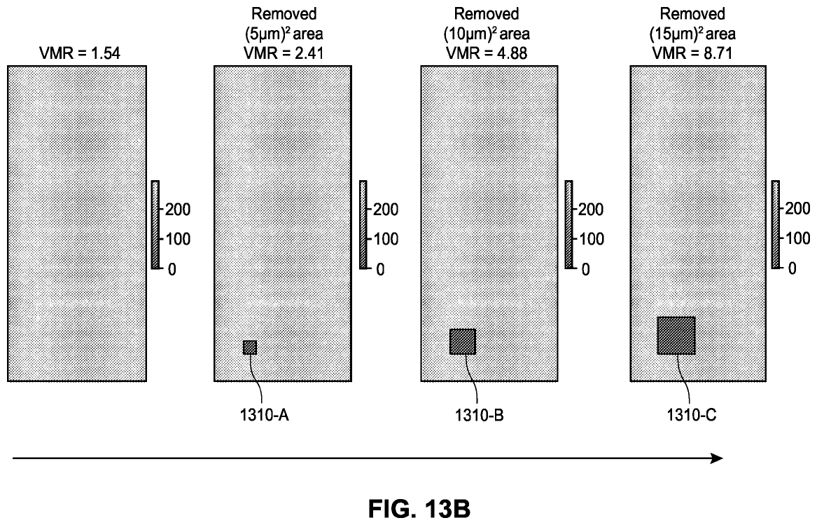

FIG. 13A depicts a heatmap of a simulated tile having a uniform distribution, according to some aspects. In particular, with a VMR of nearly 1.0, the standard deviation of the tiles is equal to the mean. In FIG. 13B, a flow cell tile (e.g., a HiSeq tile) may have areas 1310 of different sizes intentionally, progressively removed (e.g., by the quality control module 938), as a means of determining a metric to quantify variability in spatial distribution of high quality barcodes as severeness (i.e., area) of a given artifact increases. In particular, in FIG. 13B, as progressively more area is mechanically removed, the VMR grows. The following figures provide more rigorous explanation of the quantification metric for variability.

### Exemplary Computer-Implemented Quantification Metric for Variability in Spatial Distribution Aspects

FIG. 14A illustrates heatmaps 1400 and histograms 1402 illustrating cluster locations and corresponding Poisson distributions, in particular a Poisson distribution 1404 and an over-dispersed Poisson distribution 1406, according to some aspects. The Poisson distribution 1404 and the over-dispersed Poisson distribution 1406 plot a number of cluster locations (e.g., DNA fragments) per square unit of a specific area on the substrate used for sequencing. The respective VMRs of the Poisson distribution 1404 and the over-dispersed Poisson distribution 1406 are shown.

Herein, Variance Mean Ratio (VMR) is generally a normalized dispersion of count data that can be used to assess degree of uniformity in spatial distribution of random particles. In other words, VMR is a measure used to assess the dispersion or variability of data, and it is often used in the context of count data, such as data following a Poisson distribution. To calculate the VMR from a Poisson distribution, the mean can be calculated as a ratio of the mean and the variance of that distribution. The variance can be calculated (the same as the mean in Poisson distribution), in addition to the VMR, which is the ratio of the variance to the mean. In a Poisson distribution (e.g., Poisson distribution 1404), when the VMR is approximately equal to 1, (i.e., the mean and the variance are equal), it suggests that the data (e.g., spatial patterns) may follow a uniform distribution or at least has a dispersion of values around the mean that is relatively balanced and consistent. On the other hand, if spatial patterns are more irregular, VMR→∞, as shown in over-dispersed Poisson distribution 1406, or if they are more regular then VMR→0.

FIG. 14B depicts a heatmap of cluster counts and corresponding bin-wise VMR (Variance-to-Mean Ratio), according to some aspects. VMR can be computed for each tile bin-wise and averaged over all bins leading to one tile-wise value as

$$VMR = \frac{1}{n} {\sum }_{i = 1}^{n} \frac{{( {x}_{i} - \hat{x} )}^{2}}{\hat{x}} , \hat{x} = {\sum }_{i = 1}^{n} {x}_{i}$$

where n is the number of bins, xᵢ is the number of high quality barcodes in the bin and x is the average number of high quality barcodes over the n bins.

VMR may be dominated by a common spatial pattern and if too prominent, local tile artifacts can be difficult to capture. Thus, the present techniques may include computing a modified VMR (VMR_M or VMR_{M}) with respect to a normalized average tile to capture discrepancies from the common pattern as

$${VMR}_{M} = \frac{1}{n} {\sum }_{i = 1}^{n} \frac{( {x}_{i} - \hat{x} {M}_{i}^{norm} )}{\hat{x} {M}_{i}^{norm}} , {M}^{norm} = \frac{M}{\frac{1}{n} {\sum }_{i = 1}^{n} {m}_{i}} , M = \frac{1}{n} {\sum }_{j = 1}^{t} {X}_{j}$$

wherein n is the number of bins, x, is the number of high quality barcodes in bin i, \hat{x} is the average number of high quality barcodes over the n bins, M^{norm} is any spatial pattern of n bins. In some aspects, the normalized average over all tiles of the flow cell may be computed, such that xⱼ is the jth tile each consisting of n bins with the counts of high quality barcodes, t is the number of tiles, Mis the average of t tiles, mᵢ is the ith bin of M,

$${M}_{i}^{norm}$$

### is the ith bin of M^{norm}.

When computing these bin-wise, the present techniques may label bins with outlier VMR values as artifacts and count them to identify outlier tiles. In particular, the spatial data processing module 932 may compute VMR and modified VMR values based on spatial data inputs. FIG. 14C depicts heatmaps of mean density, bin-wise modified VMR, and outlier bins corresponding to FIG. 14B, according to some aspects. Herein, “outlier” bins may also be referred to as “extreme” bins.

FIGS. 14D-6H depict tile-wise metrics for spatial non-uniformity in cluster counts. FIG. 14D depicts a histogram illustrating non-uniformity of tile-wise cluster counts across flow cell(s), according to some aspects. In FIG. 14D, mean cluster counts per 20² μm² in each tile have high average values and low VMR (indicating non-uniformity in cluster counts across all tiles).

FIG. 14E depicts a histogram illustrating non-uniformity of bin-wise cluster counts across a tile, according to some aspects. In FIG. 14E, VMR quantifies overall non-uniformity in cluster counts over a single tile (recognizing that the vertical middle band of low cluster density may dominate results, and make local tile artifacts difficult to capture).

FIG. 14F depicts a histogram illustrating VMR with respect to non-uniformities apart from those found in common patterns, according to some aspects. In FIG. 14F, VMR_{M} quantifies local artifacts over a single tile (such that non-uniformities apart from the common pattern found in a majority of tiles such as the middle band is accounted for, thereby capturing discrepancies from the common pattern).

FIG. 14G depicts a histogram illustrating outlier values of bin-wise VMR_{M} per tile, according to some aspects. In FIG. 14G, the number of 20 μm² bins per tile are counted, with outlier values of VMR_{M}. FIG. 14G depicts outlier values (e.g., above a threshold of 30) of bin-wise VMR_{M} that can be used to label bins associated with an artifact.

FIG. 14H depicts a histogram illustrating tiles with outlier VMR_M values, according to some aspects. In particular, FIG. 14H depicts a percentage of tiles that have a number of outlier bins above a threshold value.

FIG. 14I depicts example histograms 1480 of tiles with substantial deviations (e.g., tiles with the highest VMR_{M}), according to some aspects. In particular FIG. 14I depicts cluster counts 1482, VMR_{M} 1484 and outlier VMR_{M} 1486.

FIG. 15A depicts heatmaps of used barcode counts and high-quality barcode counts, along with a corresponding tissue mask, and correlations of the used barcode counts and the high-quality barcode counts, according to some aspects. In particular, FIG. 15A depicts validating VMR of upstream processing by correlating it with VMR of downstream processing, to predict artifacts quantified from the downstream process. For example, a method may include computing, for all tiles in a flow cell, a spatial density of upstream high quality barcodes and downstream used barcodes, and also: a tissue/sample mask, their tile-wise VMR metrics, and for a given threshold, identifying artifacts and splitting all tiles as: artifacts in upstream and downstream processes (true positives), artifacts in the downstream process only (false negatives), artifacts in the upstream process only (false positives), or no artifacts in either upstream or downstream (true negatives). FIG. 15B depicts histograms depicting tissue masks per tile size, high-quality VMR, used count high-quality VMR greater than a threshold, and VMR used greater than the threshold corresponding to FIG. 15A, according to some aspects. FIG. 15C depicts a chart validating VMR of high-quality VMR with used VMR, according to some aspects.

FIG. 16A depicts tiles with small first VMR (Variance-to-Mean Ratio) and large second VMR, according to some aspects. FIG. 16B depicts tiles with large first VMR and large second VMR, according to some aspects. FIG. 16C depicts tiles with small first VMR and small second VMR, according to some aspects. FIG. 16D depicts a graph corresponding to FIG. 15C with labeled sub-regions corresponding respectively to FIGS. 16A-16C, according to some aspects.

FIG. 16E depicts a graph of correlation of labeled experiments, according to some aspects. In some aspects, a small number of tiles may be labeled as artifact/no-artifact by visualising used barcodes in downstream processes based on artifact tolerances determined herein. Such labels could then be used to derive thresholds for downstream VMRs, after which automatic labelling may be used by downstream VMR. This may include determining a threshold for an upstream VMR based on tiles automatically labeled by a downstream VMR, by optimizing false positive rates and true positive rates. If there are no labels, the smallest threshold at which high specificity can be guaranteed (small probability of falsely labelling as artifact) can still be determined, as shown in FIG. 16F.

For example, a threshold for labeling tiles as outlier via tile-wise VMR (e.g., 3 as in examples herein) may be derived using machine learning techniques such as logistic regression based on tile-wise labels given by an expert (e.g., an expert can review a few tiles over different flow cells and label them as artifact/no artifact). If labels are not available, the same ML techniques may be used with labels derived from corresponding tile-wise VMR computed on the bins of barcode counts used in downstream (e.g., second seq) data after exposing upstream (e.g., first seq) tiles to molecules and masking tiles with a tissue/sample mask. Tiles may then be labeled as outlier if such downstream VMR values exceed a candidate threshold. For each candidate threshold, the present techniques may compute false positive and true positive rates and choose thresholds based on desired false positive and/or true positive rates. Thresholds for labeling tiles as outlier via bin-wise VMR may include two values: 1) value of bin-wise VMR after which a bin is declared outlier (e.g., 30 in the present examples) and 2) number of outlier bins after which a tile is declared outlier (e.g., 25 in the present examples). Once the present techniques have a way to label tiles as outlier (either by expert labels or by using tile-wise VMR as explained above), the present techniques may use those labels to compute false positive and true positive rates for each candidate pair of threshold values.

FIG. 16F depicts a scatter plot illustrating minimum thresholds for high specificity of false artifact labels, according to some aspects. To generate the plot, the present techniques may include determining (e.g., via the visualization module 940) minimal VMR thresholds at which artifacts can be predicted in downstream processes from upstream processes with high confidence (thro). In some aspects, only RNA may be used instead of tissue experiments to increase correlation between upstream and downstream processes by decreasing influence of tissue morphology. Further, in some aspects, NovaSeq data may be used instead of HiSeq upstream data. Downstream artifacts may be excluded, to derive acceptable VMR thresholds (>thro). The present techniques may include investigating artifact corrections and their effects in downstream analysis. Downstream transcript counts may be normalized spatially with upstream high quality barcodes counts.

### Exemplary Computer-Implemented Method Aspects

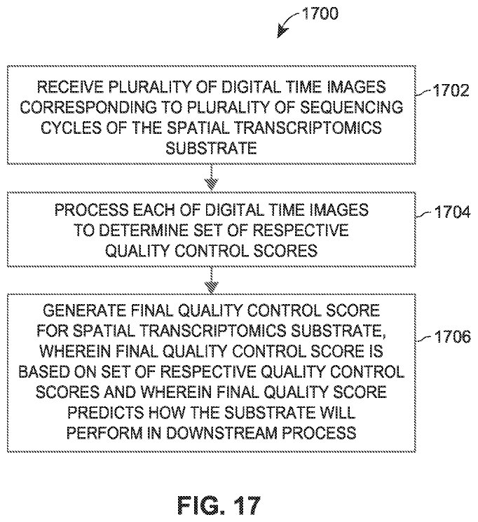

FIG. 17 depicts an exemplary computer-implemented method 1700 for improving quality control of a spatial transcriptomics substrate, according to some aspects.

The method 1700 may be implemented by the exemplary sequencing platform environment 900 in some aspects, and in particular, by the modules stored on the memory 952 of the sequencing data processing device 904. The method 1700 may include receiving, via one or more processors, a plurality of digital tile images corresponding to a plurality of sequencing cycles of the spatial transcriptomics substrate (block 1702).

The method 1700 may include processing, via one or more processors, each of the digital tile images to determine a set of respective quality control scores (block 1704).

The method 1700 may include generating, via one or more processors, a final quality control score for the spatial transcriptomics substrate (block 1706). In some aspects, the final quality control score is based on the set of respective quality control scores. In some aspects, the final quality score predicts how the substrate will perform in a downstream process. For example, in some aspects, the method may be used for performing a first seq run. Specifically, the plurality of sequencing cycles of the spatial transcriptomics substrate may correspond to a first seq sequencing step in which barcode sequences on the substrate array are read out together with their spatial coordinates. The downstream process may refer to a second seq run. Specifically, the computer-implemented aspect 1 as described below, wherein the downstream process is a second seq sequencing step wherein the substrate array of the first sequencing step is exposed to a sample whose molecules are captured and read out together with corresponding barcode sequences.

In some aspects, the spatial transcriptomics substrate corresponds to at least one of: (i) a flow cell, (i) a primary image, or (iii) a substrate containing oligos.

In some aspects, the plurality of digital tile images correspond to one or both of (i) a digital tile image of a tissue sample and (ii) a digital tile image of a fluid sample. In some aspects, the fluid sample contains at least one ribonucleic acid molecule.

In some aspects, receiving the plurality of digital tile images corresponding to the sequencing cycles of the spatial transcriptomics substrate includes receiving a plurality of barcode addresses corresponding to the spatial transcriptomics substrate, the barcode addresses each specifying a spatial positioning of a molecule located on the spatial transcriptomics substrate.

In some aspects, receiving the plurality of digital tile images corresponding to the sequencing cycles of the spatial transcriptomics substrate includes receiving at least one respective digital tissue mask corresponding to at least one of the digital tile images.

In some aspects, receiving the plurality of digital tile images corresponding to the sequencing cycles of the spatial transcriptomics substrate includes one or more of: (i) capturing one or more messenger ribonucleic acid molecules; (ii) converting one or more messenger ribonucleic acid molecules to one or more complementary deoxyribonucleic acid molecules; (iii) amplifying one or more complementary deoxyribonucleic acid molecules; or (iv) obtaining a sequence of at least one of the one or more messenger ribonucleic acid molecules by sequencing at least one of the complementary deoxyribonucleic acid molecules, wherein the sequence includes at least one barcode.

In some aspects, the method 1700 may perform bin-wise VMR computations. For example, the method 1700 may include processing each of the digital tile images to determine the set of respective quality control scores includes computing respective bin-wise variance mean ratios by: (i) subdividing at least one of the digital tile images into a plurality of bins; and (ii) for each bin in the of the plurality of bins: computing a respective bin-wise variance mean ratio for the bin. In some aspects, performing bin-wise VMR computations may include: For each bin i=1, . . . , n compute x as the number of high-quality barcodes in that bin, and it's corresponding VMR as (xᵢ−\hat{x}ᵢ)²/\hat{x}ᵢ, where \hat{x}ᵢ is the average number of HQ barcodes over all n bins in a tile, i.e., \hat{x}ᵢ=sumᵢxᵢ. Here the average value doesn't have to be strictly restricted to a tile, but potentially a subtile region of n bins could be used. The method 1700 may include counting high quality barcodes. The threshold 30 may be used, but potentially one can use a different threshold: it may be chosen to maintain a correlation between upstream and downstream processing.

In some aspects, the method 1700 may perform tile-wise VMR computations. For example, the method 1700 may include computing at least one tile-wise average variance mean ratio y counting HQ barcodes in each tile y_i over all m tiles and then using the same formula as above with y instead of x and m instead of n according to:

$$VMR = \frac{1}{n} {\sum }_{i = 1}^{n} \frac{{( {x}_{i} - \hat{x} )}^{2}}{\hat{x}} , \hat{x} = {\sum }_{i = 1}^{n} {x}_{i} ,$$

where n is the number of bins, xᵢ is the number of high quality barcodes in the bin and \hat{x} is the average number of high quality barcodes over the n bins.

In some aspects, the method 1700 may perform flow cell-wise VMR computations. For example, the method 1700 may include computing a substrate-wise variance mean ratio by averaging the respective tile-wise average variance mean ratio of each of the plurality of bins.

In some aspects, the method 1700 may perform tile-wise modified VMR computations. For example, the method 1700 may include computing at least one tile-wise average variance mean ratio by averaging normalized tile bin values, according to

$${VMR}_{M} = \frac{1}{n} {\sum }_{i = 1}^{n} \frac{( {x}_{i} - \hat{x} {M}_{i}^{norm} )}{\hat{x} {M}_{i}^{norm}} , {M}^{norm} = \frac{M}{\frac{1}{n} {\sum }_{i = 1}^{n} {m}_{i}} , M = \frac{1}{t} {\sum }_{j = 1}^{t} {X}_{j} ,$$

wherein n is the number of bins, xᵢ is the number of high quality barcodes in bin i, \hat{x} is the average number of high quality barcodes over the n bins, M^{norm} is any spatial pattern of n bins, xⱼ is the jth tile each consisting of n bins with the counts of high quality barcodes, t is the number of tiles, Mis the average of t tiles, mᵢ is the ith bin of M, and

$${M}_{i}^{norm}$$

### is the ith bin of M^{norm}.

In some aspects, the method 1700 may include labeling outlier bins. For example, the method 1700 may include computing the respective bin-wise variance mean ratio for the bin includes: (i) comparing the respective bin-wise variance mean ratio to a bin threshold value; and (ii) when the respective bin-wise variance mean ratio exceeds the bin threshold value, labeling the bin as an outlier bin. In some aspects, the method 1700 may include labeling outlier tiles based on a tile-wise VMR value: if a tile exceeds a given threshold, label tile as outlier with respect to tile-wise VMR.

In some aspects, the method 1700 may include labeling outlier tiles. For example, the method 1700 may include (i) determining the number of labeled outlier bins in at least one of the plurality of bins; and (ii) when the number of labeled outlier bins exceeds a tile threshold value, labeling the tile as an outlier tile.

In some aspects, the method 1700 may include predicting an artifact in a second spatial transcriptomics substrate based at least in part on the quality control scores, or another aspect of the method 1700 with respect to the spatial transcriptomics substrate.

In some aspects, the method 1700 may include determining whether the spatial transcriptomics substrate is usable based at least in part on the quality control scores. For example, the method 1700 may be used to avoid shipping defective first seq flow cells, in some aspects. In some aspects, the method 1700 may be used to avoid other potentially undesirable uses of first seq flow cells or other spatial transcriptomic substrates, such as using them for experimental purposes.

In some aspects, the method 1700 may include normalizing data in the downstream process.

In some aspects, the method 1700 may include correlating barcode counts of the spatial transcriptomics substrate with barcode counts of the downstream process.

In some aspects, the method 1700 may include generating a visualization of at least one of: (i) the set of respective quality control scores; (ii) VMR of a flow cell of the substrate; (iii) VMR of a tile of the substrate; (iv) VMR with respect to M per tile of the substrate; (v) a number of outlier VMR values of bins in a tile of the substrate; or (vi) a tile having more than a threshold number of bins with outlier VMR_{M} values.

In some aspects, the method 1700 may include generating a heatmap, a histogram, a plot or a line chart.

### ADDITIONAL CONSIDERATIONS

Although the disclosure herein sets forth a detailed description of numerous different implementations, it should be understood that the legal scope of the description is defined by the words of the claims set forth at the end of this patent and equivalents. The detailed description is to be construed as exemplary only and does not describe every possible implementation since describing every possible implementation would be impractical. Numerous alternative implementations may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein, an element or step recited in the singular and proceeded with the word “a” or “an” should be understood as not excluding plural of said elements or steps, unless such exclusion is explicitly stated. Furthermore, references to “one implementation” are not intended to be interpreted as excluding the existence of additional implementations that also incorporate the recited features. Moreover, unless explicitly stated to the contrary, implementations “comprising,” “including,” or “having” an element or a plurality of elements having a particular property may include additional elements whether or not they have that property. Moreover, the terms “comprising,” including, “having,” or the like are interchangeably used herein.

The terms “substantially,” “approximately,” and “about” used throughout this Specification are used to describe and account for small fluctuations, such as due to variations in processing. For example, they can refer to less than or equal to +5%, such as less than or equal to ±2%, such as less than or equal to ±1%, such as less than or equal to ±0.5%, such as less than or equal to ±0.2%, such as less than or equal to 0.1%, such as less than or equal to ±0.5%. In one example, these terms include situation where there is no variation—0%.

Underlined and/or italicized headings and subheadings are used for convenience only, do not limit the subject technology, and are not referred to in connection with the interpretation of the description of the subject technology. All structural and functional equivalents to the elements of the various implementations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality and improve the functioning of conventional computers.

Additionally, certain implementations are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example implementations, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example implementations, comprise processor-implemented modules.

Similarly, the methods or routines described herein may be at least partially processor implemented. For example, at least some of the operations of a method may be performed by one or more processors or processor-implemented hardware modules. The performance of certain of the operations may be distributed among the one or more processors, not only residing within a single machine, but deployed across a number of machines. In some example implementations, the processor or processors may be located in a single location, while in other implementations the processors may be distributed across a number of locations.

The performance of certain of the operations may be distributed among the one or more processors, not only residing within a single machine, but deployed across a number of machines. In some example implementations, the one or more processors or processor-implemented modules may be located in a single geographic location (e.g., within a home environment, an office environment, or a server farm). In other implementations, the one or more processors or processor-implemented modules may be distributed across a number of geographic locations.

A person of ordinary skill in the art may implement numerous alternate implementations, using either current technology or technology developed after the filing date of this application. Those of ordinary skill in the art will recognize that a wide variety of modifications, alterations, and combinations may be made with respect to the above described implementations without departing from the scope of the invention, and that such modifications, alterations, and combinations are to be viewed as being within the ambit of the inventive concept.

By way of example, and not limitation, the disclosure herein contemplates at least the following aspects.

Aspect 1. A computer-implemented method for improving quality control of a spatial transcriptomics substrate, the method comprising: receiving, via one or more processors, a plurality of digital tile images corresponding to a plurality of sequencing cycles of the spatial transcriptomics substrate; processing, via one or more processors, each of the digital tile images to determine a set of respective quality control scores; and generating, via one or more processors, a final quality control score for the spatial transcriptomics substrate, wherein the final quality control score is based on the set of respective quality control scores, and wherein the final quality score predicts how the substrate will perform in a downstream process.

Aspect 2. The computer-implemented method of aspect 1, wherein the plurality of sequencing cycles of the spatial transcriptomics substrate corresponds to a first seq sequencing step in which barcode sequences on a substrate array of the spatial transcriptomics substrate are read out together with their spatial coordinates.

Aspect 3. The computer-implemented method of any of the preceding aspects, wherein the downstream process is a second seq sequencing step wherein the substrate array of the first seq sequencing step is exposed to a sample whose molecules are captured and read out together with corresponding barcode sequences.

Aspect 4. The computer-implemented method of any of the preceding aspects, wherein the downstream process is a second seq sequencing step wherein the substrate array of the first seq sequencing step is exposed to a sample whose molecules are captured and read out together with corresponding barcode sequences.

Aspect 5. The computer-implemented method of any of the preceding aspects, wherein the plurality of digital tile images correspond to one or both of (i) a digital tile image of a tissue sample and (ii) a digital tile image of a fluid sample.

Aspect 6. The computer-implemented method of aspect 5, wherein the fluid sample contains at least one ribonucleic acid molecule.

Aspect 7. The computer-implemented method of any of the preceding aspects, wherein receiving the plurality of digital tile images corresponding to the sequencing cycles of the spatial transcriptomics substrate includes receiving a plurality of barcode addresses corresponding to the spatial transcriptomics substrate, the barcode addresses each specifying a spatial positioning of a molecule located on the spatial transcriptomics substrate.

Aspect 8. The computer-implemented method of any of the preceding aspects, wherein receiving the plurality of digital tile images corresponding to the sequencing cycles of the spatial transcriptomics substrate includes receiving at least one respective digital tissue mask corresponding to at least one of the digital tile images.

Aspect 9. The computer-implemented method of any of the preceding aspects, wherein receiving the plurality of digital tile images corresponding to the sequencing cycles of the spatial transcriptomics substrate includes one or more of: capturing one or more messenger ribonucleic acid molecules; converting one or more messenger ribonucleic acid molecules to one or more complementary deoxyribonucleic acid molecules; amplifying one or more complementary deoxyribonucleic acid molecules; or obtaining a sequence of at least one of the one or more messenger ribonucleic acid molecules by sequencing at least one of the complementary deoxyribonucleic acid molecules, wherein the sequence includes at least one barcode.

Aspect 10. The computer-implemented method of any of the preceding aspects, wherein processing each of the digital tile images to determine the set of respective quality control scores includes computing respective bin-wise variance mean ratios by: subdividing at least one of the digital tile images into a plurality of bins; and for each bin in the of the plurality of bins: computing a respective bin-wise variance mean ratio for the bin.

Aspect 11. The computer-implemented method of aspect 10, further comprising: computing at least one tile-wise average variance mean ratio (VMR) by averaging the respective bin-wise variance mean ratios of the plurality of bins, according to

$$VMR = \frac{1}{n} {\sum }_{i = 1}^{n} \frac{{( {x}_{i} - \hat{x} )}^{2}}{\hat{x}} , \hat{x} = {\sum }_{i = 1}^{n} {x}_{i} ,$$

where n is the number of bins, xᵢ is the number of high quality barcodes in the bin and z is the average number of high quality barcodes over the n bins.

Aspect 12. The computer-implemented method of aspect 11, further comprising: computing a substrate-wise variance mean ratio by averaging the respective tile-wise average variance mean ratio of each of the plurality of bins.

Aspect 13. The computer-implemented method of aspect 10, further comprising: computing at least one tile-wise average variance mean ratio (VMR_{M}) by averaging normalized tile bin values, according to

$${VMR}_{M} = \frac{1}{n} {\sum }_{i = 1}^{n} \frac{( {x}_{i} - \hat{x} {M}_{i}^{norm} )}{\hat{x} {M}_{i}^{norm}} , {M}^{norm} = \frac{M}{\frac{1}{n} {\sum }_{i = 1}^{n} {m}_{i}} , M = \frac{1}{t} {\sum }_{i = 1}^{t} {X}_{j} ,$$

wherein n is the number of bins, xᵢ is the number of high quality barcodes in bin i, \hat{x} is the average number of high quality barcodes over the n bins, M^{norm} is any spatial pattern of n bins, xⱼ is the jth tile each consisting of n bins with the counts of high quality barcodes, t is the number of tiles, Mis the average of t tiles, mᵢ is the ith bin of M, and

$${M}_{i}^{norm}$$

### is the ith bin of M^{norm}.

Aspect 14. The computer-implemented method of aspect 10, wherein computing the respective bin-wise variance mean ratio for the bin includes: comparing the respective bin-wise variance mean ratio to a bin threshold value; and based at least in part on the respective bin-wise variance mean ratio meeting the bin threshold value, labelling the bin as an outlier bin.

Aspect 15. The computer-implemented method of aspect 14, further comprising: determining a number of labeled outlier bins in at least one of the plurality of bins; and based at least in part on the number of labeled outlier bins meeting a tile threshold value, labelling the tile as an outlier tile.

Aspect 16. The computer-implemented method of any of the preceding aspects, further comprising: predicting an artifact in a second spatial transcriptomics substrate based at least in part on the quality control scores.

Aspect 17. The computer-implemented method of any of the preceding aspects, further comprising: determining whether the spatial transcriptomics substrate is usable based at least in part on the quality control scores.

Aspect 18. The computer-implemented method of any of the preceding aspects, further comprising: normalizing data in the downstream process.

Aspect 19. The computer-implemented method of any of the preceding aspects, further comprising: correlating one or more barcode counts of the spatial transcriptomics substrate with barcode counts of the downstream process.

Aspect 20. The computer-implemented method of any of the preceding aspects, further comprising: generating a visualization of at least one of: (i) the set of respective quality control scores; (ii) a variance mean ratio (VMR) of a flow cell of the substrate; (iii) a VMR of a tile of the substrate; (iv) a VMR with respect to M per tile of the substrate; (v) a number of outlier VMR values of bins in a tile of the substrate; or (vi) a tile having more than a threshold number of bins with outlier variance mean ratio with respect to M (VMR_{M}) values.

Aspect 21. The computer-implemented method of aspect 20, wherein the visualization is a heatmap, a histogram, a plot, or a line chart.

Aspect 22. A computing system for improving quality control of a spatial transcriptomics substrate, comprising: one or more processors; and one or more memories having stored thereon computer-executable instructions that, when executed, cause the computing system to: receive a plurality of digital tile images corresponding to a plurality of sequencing cycles of the spatial transcriptomics substrate; process each of the digital tile images to determine a set of respective quality control scores; and generate a final quality control score for the spatial transcriptomics substrate, wherein the final quality control score is based on the set of respective quality control scores, and wherein the final quality score predicts how the substrate will perform in a downstream process.

Aspect 23. A non-transitory computer-readable medium having stored thereon computer-executable instructions that, when executed, cause a computer to: receive a plurality of digital tile images corresponding to a plurality of sequencing cycles of a spatial transcriptomics substrate; process each of the digital tile images to determine a set of respective quality control scores; and generate a final quality control score for the spatial transcriptomics substrate, wherein the final quality control score is based on the set of respective quality control scores, and wherein the final quality score predicts how the substrate will perform in a downstream process.

Aspect 24. A computing system for quality control of spatial analysis products in a spatial genomics workflow, comprising: a processor; and a memory having stored thereon computer-executable instructions that, when executed by the processor, cause the computing system to: compute quality control metrics with respect to flow cells; predict second seq quality from first seq data; detect non-uniformities on a substrate; generate tile-wise, bin-wise, sample-wise, and substrate-wise scores using predetermined threshold values; and control quality of upstream spatial analysis products based on the generated scores.

Aspect 25. The computing system of aspect 24, wherein the flow cells include uniform/random flow cells and patterned flow cells.

Aspect 26. The computing system of aspect 24 or aspect 25, wherein the quality control metrics include variance-based statistical measures.

Aspect 27. The computing system of any of aspects 24 to 26, wherein the first seq comprises reading out barcode sequences on a substrate array together with their spatial coordinates.

Aspect 28. The computing system of any of aspects 24 to 27, wherein the second seq comprises exposing the substrate array of the first seq to a sample, capturing molecules, and reading out the captured molecules together with corresponding barcode sequences.

Aspect 29. The computing system of any of aspects 24 to 28, wherein controlling quality of upstream spatial analysis products comprises adjusting the predetermined threshold values.

Aspect 30. The computing system of any of aspects 24 to 29, further comprising a sequencing platform.

What is claimed is:

1. A method for stitching genomic sequence data, the method comprising:
obtaining, by one or more processors, tile data for respective tiles in a pair of overlapping swaths of genomic sequence data, wherein the tile data is a dataset corresponding to a region of a sample, the dataset including a coordinate system and a set of reads of genomic sequences at locations within the coordinate system;
for respective pairs of adjacent tiles in the pair of overlapping swaths:
(i) identifying, by the one or more processors, one or more matching reads at respective locations within the pair of adjacent tiles; and
(ii) determining, by the one or more processors, a pairwise offset for the pair of adjacent tiles based on the respective locations of the one or more matching reads;
determining, by the one or more processors, a swath offset for the pair of overlapping swaths based on the pairwise offsets for the respective pairs of adjacent tiles in the pair of overlapping swaths; and
stitching, by the one or more processors, the respective pairs of adjacent tiles together by aligning the coordinate systems of the respective tiles using the swath offset to generate a continuous coordinate system across the pair of overlapping swaths.

2. The method of claim 1, wherein the pair of overlapping swaths includes a first swath and a second swath, and wherein stitching respective pairs of adjacent tiles together includes:
applying, by the one or more processors, a transformation to the coordinate systems of tiles in the second swath based on the swath offset;
determining, by the one or more processors, a middle portion of the continuous coordinate system between the first and second swaths; and
filtering, by the one or more processors, one or more duplicated reads from the continuous coordinate system, wherein a duplicated read is at a location within the continuous coordinate system on an opposite side of the middle portion from the first or second swath where the duplicated read was identified before the transformation.

3. The method of claim 1, wherein respective tiles within the pair of swaths are overlapping, wherein the pair of overlapping swaths are adjacent horizontally such that respective pairs of adjacent tiles in the pair of overlapping swaths are adjacent horizontally, the pairwise offset is a horizontal pairwise offset, and the swath offset is a horizontal swath offset, and further comprising:
for respective pairs of vertically adjacent tiles within a particular swath:
(i) identifying, by the one or more processors, one or more matching reads at respective locations within the pair of vertically adjacent tiles; and
(ii) determining, by the one or more processors, a vertical pairwise offset for the pair of vertically adjacent tiles based on the respective locations of the one or more matching reads;
determining, by the one or more processors, a vertical swath offset for the particular swath based on the vertical pairwise offsets for the respective pairs of vertically adjacent tiles in the particular swath; and
stitching, by the one or more processors, respective pairs of vertically adjacent tiles together by aligning the coordinate systems of respective tiles using the vertical swath offset.

4. The method of claim 3, further comprising:
rotating, by the one or more processors, a coordinate system for at least one of the respective tiles within the particular swath to align the coordinate system of the tile with coordinate systems for other tiles within the particular swath.

5. The method of claim 4, wherein respective tiles are generated for a particular cycle of a plurality of cycles, and wherein rotating the coordinate system for a respective tile includes:
selecting, by the one or more processors, a desired cycle for use in aligning the coordinate systems of the respective tiles; and
aligning, by the one or more processors, the coordinate systems of the respective tiles to a coordinate system for the desired cycle by applying an affine transformation for the desired cycle.

6. The method of claim 5, wherein aligning the coordinate systems of the respective tiles further includes:
for the respective tiles:
determining, by the one or more processors, for the desired cycle, one or more of: (i) a translation parameter, (ii) a magnification parameter, or (iii) a shear mapping parameter;
determining, by the one or more processors, a center location of the respective tile;
identifying, by the one or more processors, locations of clusters of reads within the respective tile; and
for respective cluster locations, applying, by the one or more processors, to a difference between the respective cluster location and the center location, one or more of: (i) the translation parameter, (ii) the magnification parameter, or (iii) the shear mapping parameter.

7. The method of claim 1, further comprising at least one of:
identifying, by the one or more processors, a genomic sequence at a particular location using the continuous coordinate system;
identifying, by the one or more processors, a genetic variant based on the genomic sequence; or
identifying, by the one or more processors, a phenotype associated with the genetic variant.

8. A system for stitching genomic sequence data, the system comprising:
one or more processors;
one or more memories coupled to the one or more processors; and
computer-readable instructions stored in the one or more memories that, when executed by the one or more processors, cause the system to:
obtain tile data for respective tiles in a pair of overlapping swaths of genomic sequence data, wherein the tile data is a dataset corresponding to a region of a sample, the dataset including a coordinate system and a set of reads of genomic sequences at locations within the coordinate system,
for respective pairs of adjacent tiles in the pair of overlapping swaths:
(i) identify one or more matching reads at respective locations within the pair of adjacent tiles, and
(ii) determine a pairwise offset for the pair of adjacent tiles based on the respective locations of the one or more matching reads,
determine a swath offset for the pair of overlapping swaths based on the pairwise offsets for the respective pairs of adjacent tiles in the pair of overlapping swaths, and
stitch the respective pairs of adjacent tiles together by aligning the coordinate systems of the respective tiles using the swath offset to generate a continuous coordinate system across the pair of overlapping swaths.

9. The system of claim 8, wherein the pair of overlapping swaths includes a first swath and a second swath, and wherein to stitch respective pairs of adjacent tiles together, the computer-readable instructions cause the system to:
apply a transformation to the coordinate systems of tiles in the second swath based on the swath offset;
determine a middle portion of the continuous coordinate system between the first and second swaths; and
filter one or more duplicated reads from the continuous coordinate system, wherein a duplicated read is at a location within the continuous coordinate system on an opposite side of the middle portion from the first or second swath where the duplicated read was identified before the transformation.

10. The system of claim 8, wherein respective tiles within the pair of swaths are overlapping, wherein the pair of overlapping swaths are adjacent horizontally such that respective pairs of adjacent tiles in the pair of overlapping swaths are adjacent horizontally, the pairwise offset is a horizontal pairwise offset, the swath offset is a horizontal swath offset, and the computer-readable instructions further cause the system to:
for respective pairs of vertically adjacent tiles within a particular swath:
(i) identify one or more matching reads at respective locations within the pair of vertically adjacent tiles, and
(ii) determine a vertical pairwise offset for the pair of vertically adjacent tiles based on the respective locations of the one or more matching reads,
determine a vertical swath offset for the particular swath based on the vertical pairwise offsets for the respective pairs of vertically adjacent tiles in the particular swath, and
stitch the respective pairs of vertically adjacent tiles together by aligning the coordinate systems of the respective tiles using the vertical swath offset.

11. The system of claim 10, wherein the computer-readable instructions further cause the system to:
rotate a coordinate system for at least one of the respective tiles within the particular swath to align the coordinate system of the tile with coordinate systems for other tiles within the particular swath.

12. The system of claim 11, wherein respective tiles are generated for a particular cycle of a plurality of cycles, and wherein to rotate the coordinate system for a respective tile, the instructions cause the system to:
select a desired cycle for use in aligning the coordinate systems of the respective tiles, and
align the coordinate systems of the respective tiles to a coordinate system for the desired cycle by applying an affine transformation for the desired cycle.

13. The system of claim 12, wherein to align the coordinate systems of the respective tiles, the instructions further cause the system to:
for the respective tiles:
determine, for the desired cycle, one or more of: (i) a translation parameter, (ii) a magnification parameter, or (iii) a shear mapping parameter;
determine a center location of the respective tile;
identify locations of clusters of reads within the respective tile; and
for respective cluster locations, apply, to a difference between the respective cluster location and the center location, one or more of: (i) the translation parameter, (ii) the magnification parameter, or (iii) the shear mapping parameter.

14. The system of claim 8, wherein the instructions further cause the system to at least one of:
identify a genomic sequence at a particular location using the continuous coordinate system;
identify a genetic variant based on the genomic sequence; or
identify a phenotype associated with the genetic variant.

15. A tangible, non-transitory computer-readable medium storing instructions for stitching genomic sequence data that, when executed by one or more processors, cause the one or more processors to:
obtain tile data for respective tiles in a pair of overlapping swaths of genomic sequence data, wherein the tile data is a dataset corresponding to a region of a sample, the dataset including a coordinate system and a set of reads of genomic sequences at locations within the coordinate system,
for respective pairs of adjacent tiles in the pair of overlapping swaths:
(i) identify one or more matching reads at respective locations within the pair of adjacent tiles, and
(ii) determine a pairwise offset for the pair of adjacent tiles based on the respective locations of the one or more matching reads,
determine a swath offset for the pair of overlapping swaths based on the pairwise offsets for the respective pairs of adjacent tiles in the pair of overlapping swaths, and
stitch the respective pairs of adjacent tiles together by aligning the coordinate systems of the respective tiles using the swath offset to generate a continuous coordinate system across the pair of overlapping swaths.

16. The computer-readable medium of claim 15, wherein the pair of overlapping swaths includes a first swath and a second swath, and wherein to stitch respective pairs of adjacent tiles together, the instructions cause the one or more processors to:
apply a transformation to the coordinate systems of tiles in the second swath based on the swath offset;
determine a middle portion of the continuous coordinate system between the first and second swaths; and
filter one or more duplicated reads from the continuous coordinate system, wherein a duplicated read is at a location within the continuous coordinate system on an opposite side of the middle portion from the first or second swath where the duplicated read was identified before the transformation.

17. The computer-readable medium of claim 15, wherein respective tiles within the pair of swaths are overlapping, wherein the pair of overlapping swaths are adjacent horizontally such that respective pairs of adjacent tiles in the pair of overlapping swaths are adjacent horizontally, the pairwise offset is a horizontal pairwise offset, the swath offset is a horizontal swath offset, and the instructions further cause the one or more processors to:
for respective pairs of vertically adjacent tiles within a particular swath:
(i) identify one or more matching reads at respective locations within the pair of vertically adjacent tiles, and
(ii) determine a vertical pairwise offset for the pair of vertically adjacent tiles based on the respective locations of the one or more matching reads,
determine a vertical swath offset for the particular swath based on the vertical pairwise offsets for the respective pairs of vertically adjacent tiles in the particular swath, and
stitch the respective pairs of vertically adjacent tiles together by aligning the coordinate systems of the respective tiles using the vertical swath offset.

18. The computer-readable medium of claim 17, wherein the instructions further cause the one or more processors to:
rotate a coordinate system for at least one of the respective tiles within the particular swath to align the coordinate system of the tile with coordinate systems for other tiles within the particular swath.

19. The computer-readable medium of claim 18, wherein respective tiles are generated for a particular cycle of a plurality of cycles, and wherein to rotate the coordinate system for the tile, the instructions cause the one or more processors to:
select a desired cycle for use in aligning the coordinate systems of the respective tiles, and
align the coordinate systems of the respective tiles to a coordinate system for the desired cycle by applying an affine transformation for the desired cycle.

20. The computer-readable medium of claim 19, wherein to align the coordinate systems of respective tiles, the instructions further cause the one or more processors to:
for the respective tiles:
determine, for the desired cycle, one or more of: (i) a translation parameter, (ii) a magnification parameter, or (iii) a shear mapping parameter;
determine a center location of the respective tile;
identify locations of clusters of reads within the respective tile; and
for respective cluster locations, apply, to a difference between the respective cluster location and the center location, one or more of: (i) the translation parameter, (ii) the magnification parameter, or (iii) the shear mapping parameter.
